(19) United States
(12) Patent Application Publication
(10) Pub. No.: US 2012/0109019 A1
(43) Pub. Date: May 3, 2012

(54) APPARATUS AND METHOD FOR ULTRASONIC SPINE TREATMENT

(75) Inventors: John B. Schneider, Memphis, TN (US); F. Javier De Ana, Chapel Hill, NC (US); Neill M. Pounder, Cary, NC (US); Kevin J. Tanis, Collierville, TN (US); Jin (NMI) Zhang, Memphis, TN (US); Craig P. Conner, Madison, WI (US); Daniel R. Bullis, Madison, WI (US); Mark W. Cors, Columbus, TN (US); Daniel J. Lee, Monticello, WI (US); Rachel E. Desmidt, Madison, WI (US); Nicholas S. Reback, Monona, WI (US); Stephen A. Latham, Sun Prairie, WI (US)

(73) Assignee: SMITH & NEPHEW, INC., Memphis, TN (US)

(21) Appl. No.: 13/126,511

(22) PCT Filed: Oct. 29, 2009
(86) PCT No.: PCT/US09/62542

§ 371 Date: Jul. 12, 2011

Related U.S. Application Data
(60) Provisional application No. 61/109,824, filed on Oct. 30, 2008.

(51) Int. Cl. A61N 7/00

(52) U.S. Cl. 601/2

(57) ABSTRACT

The present invention relates to an apparatus for therapeutically treating bone structure using ultrasound, and more particularly, the present invention relates to an apparatus with an attachment structure for treating bone injuries or a variety of musculoskeletal injuries and/or problems.

### CROSS-REFERENCE TO RELATED APPLICATIONS

The present invention claims priority to US provisional application No. 61/109824 entitled “Apparatus and methods for ultrasonic spine treatment” filed on 30 Oct. 2008, which is herein incorporated in its entirety.

### FIELD OF THE INVENTION

The present invention relates to an apparatus for therapeutically treating bone structure using ultrasound, and more particularly, the present invention relates to an apparatus with an attachment structure for treating bone injuries or a variety of musculoskeletal injuries and/or problems.

### BACKGROUND OF THE INVENTION

The use of ultrasound to therapeutically treat and evaluate bone injuries is known. Impinging ultrasonic pulses having appropriate parameters, e.g., frequency, pulse repetition, and amplitude, for suitable periods of time and at a proper external location adjacent to a bone injury has been determined to accelerate the natural healing of, for example, bone breaks and fractures. For patients with reduced healing capacity, such as elderly persons with osteoporosis, ultrasonic therapy may promote healing of bone injuries that would otherwise require prosthetic replacement or leave the patient permanently disabled.

U.S. Pat. No. 4,530,360 to Duarte describes a basic non-invasive therapeutic technique and apparatus for applying ultrasonic pulses from an operative surface placed on the skin at a location adjacent a bone injury. The applicator described in the '360 patent has a plastic tube which serves as a grip for the operator, an RF plug attached to the plastic tube for connection to an RF source, and internal cabling connected to an ultrasonic transducer. To apply the ultrasound pulses during treatment an operator must manually hold the applicator in place until the treatment is complete. As a result, the patient is, in effect, immobilized during treatment. The longer the treatment period, the more the patient and/or the assistant is inconvenienced, and certain parts of the body, such as the back, cannot be reached by the patient with such a device, thus requiring the help of an assistant. The '360 patent also describes a range of RF signals for creating the ultrasound, ultrasound power density levels, a range of duration for each ultrasonic pulse, and a range of ultrasonic pulse frequencies.

U.S. Pat. No. 5,003,965 to Talish et al. relates to an ultrasonic body treatment system having a body-applicator unit connected to a remote control unit by sheathed fiber optic lines. The signals controlling the duration of ultrasonic pulses and the pulse repetition frequency are generated apart from the body-applicator unit. Talish et al. also describes a mounting fixture that is adapted for use with a cast for attaching the body-applicator unit to a patient so that the operative surface is adjacent the skin location.

U.S. Pat. No. 5,211,160 to Talish et al. relates to an ultrasonic treatment system with a mounting fixture that attaches to a patient's limb using straps and a hook and loop attachment. The body application unit interfaces with the mounting fixture so that the operative surface is adjacent to the skin location.

While the systems described in these patents relate to therapeutic methods and apparatus for ultrasonically treating injured bone, and describe basic mounting fixtures for use with a cast or limb for attaching the body applicator unit to the patient, they do not disclose signal generators and transducers, and attachments therefore, that permit placement of the body-applicator unit adjacent to various parts of the body that are either hard-to-reach or, because of the topology of the external skin location, make it difficult to manually position and maintain a transducer adjacent thereto. Nor do these systems permit patient mobility during treatment.

Spinal fusion, also known as spondylodesis or spondylosyndesis, is a surgical technique used to combine two or more vertebrae. Supplementary bone tissue (either autograft or allograft) is used in conjunction with the body's natural osteoblastic processes. This procedure is used primarily to eliminate the pain caused by abnormal motion of the vertebrae by immobilizing the vertebrae themselves. Spinal fusion is done most commonly in the lumbar region of the spine, but it is also used to treat cervical and thoracic problems. People rarely have problems with the thoracic spine because there is little normal motion in the thoracic spine. Patients requiring spinal fusion have either neurological deficits or severe pain which has not responded to conservative treatment.

There are two main types of lumbar spinal fusion, which may be used in conjunction with each other: Posterolateral fusion and Interbody fusion. Posterolateral fusion places the bone graft between the transverse processes in the back of the spine. These vertebrae are then fixed in place with screws and/or wire through the pedicles of each vertebra attaching to a metal rod on each side of the vertebrae.

Interbody fusion places the bone graft between the vertebra in the area usually occupied by the intervertebral disc. In preparation for the spinal fusion, the disc is removed entirely. A device may be placed between the vertebra to maintain spine alignment and disc height. The intervertebral device may be made from either plastic or titanium. The fusion then occurs between the endplates of the vertebrae. Two primary types of interbody fusion are: Anterior lumbar interbody fusion (ALIF) and Posterior lumbar interbody fusion (PLIF). In ALIF, an anterior abdominal incision is used to reach the lumbar spine, and in PLIF a posterior incision is used to reach the lumbar spine. Another type of fusion is Transforaminal Lumbar Interbody Fusion (TLIF). TLIF fuses the anterior (front) and posterior (back) columns of the spine through a single posterior approach.

In most cases, the fusion is augmented by a process called fixation, meaning the placement of metallic screws (pedicle screws often made from titanium), rods or plates, or cages to stabilize the vertebra to facilitate bone fusion. The fusion process typically takes 6-12 months after surgery. During in this time external bracing (orthotics) may be required. External factors such as smoking, osteoporosis, certain medications, and heavy activity can prolong or even prevent the fusion process. If fusion does not occur, patients may require re-operation.

Therefore, a need exists for apparatus which optimize healing while maintaining patient mobility. In particular, a need exists for an apparatus which permits placement of the body-applicator unit adjacent to various parts of the body that are hard-to-reach or otherwise hard to manually position a transducer adjacent thereto, such as the spine, hip, or pelvis region.

### SUMMARY OF THE INVENTION

According to a first aspect of the invention there is provided an ultrasound delivery system for use in accelerating bone formation in a subject, the system comprising a subject attachment structure having a subject-facing surface which is configured to substantially contour to a region of the subject to which ultrasound is to be delivered, the subject attachment structure comprising;
a pair of ultrasound transducer holders located on the subject-facing surface, each holder capable of receiving an ultrasound transducer,
a belt attachment region.

In embodiments of the invention the subject attachment structure is provided with a mechanism for altering at least one of the superior/inferior, the medial/lateral, or the anterior/posterior position of at least one transducer holder of the pair relative to the region of the subject to which the subject attachment structure is applied.

In embodiments of the invention the transducer holders are removably locatable on the subject attachment structure

In embodiments of the invention the subject attachment structure is provided with an array of transducer holder positioning holes to which the transducer holders are securable.

In embodiments of the invention at least one transducer holder of the pair is carried on a transducer holder carrying member and wherein the transducer holder carrying member is associated with and moveable along a track which extends at least partially across the subject-facing surface.

In embodiments of the invention each of the transducer holders of the pair is carried on the transducer holder carrying member.

In embodiments of the invention each transducer holder extends from the transducer holder carrying member in an opposing direction.

In embodiments of the invention the transducer holder carrying member extends between two substantially parallel tracks.

In embodiments of the invention each of the transducer holders of the pair is carried on a separate transducer holder carrying member, and wherein each transducer holder carrying member is associated with and moveable along a track.

In embodiments of the invention the transducer holder carrying members are movable along the same track.

In embodiments of the invention the transducer holder carrying members are independently moveable along the track.

In embodiments of the invention each transducer holder carrying member is associated with a separate track.

In embodiments of the invention the separate tracks are substantially parallel.

In embodiments of the invention the separate tracks are substantially perpendicular.

In embodiments of the invention the transducer holder carrying member is slidably attachable to the track.

In embodiments of the invention the subject attachment structure is provided with a peripheral frame and wherein the track extends between opposing edges of the frame.

In embodiments of the invention the track forms opposing edges of the frame.

In embodiments of the invention the track is a rail which extends outwardly from the subject-facing surface.

In embodiments of the invention the transducer holder or at least one transducer holder of a pair is moveable on the transducer holder carrying member.

In embodiments of the invention the transducer holder is slidably moveable on the transducer holder carrying member.

In embodiments of the invention the transducer holders are capable of being adjusted in an anterior/posterior direction relative to the subject when the subject attachment structure is in situ.

In embodiments of the invention at least one of the transducer holder or the transducer holder carrying member is provided with a mechanism to enable at least of one of the angular or rotational adjustment of the transducer holder.

In embodiments of the invention wherein a locking mechanism is provided to lock the transducer holder in a specific orientation.

In embodiments of the invention the angle of the transducer holder can be adjusted to be from about 1° to about 50° relative to the subject-facing surface.

In embodiments of the invention the angle is from about 1° to about 35° relative to the subject-facing surface.

In embodiments of the invention the transducer holder comprises a dish or cup portion adapted to receive a transducer.

In embodiments of the invention a gel retaining cup is associated with the transducer holder.

In embodiments of the invention the subject attachment structure comprises at least two pairs of transducer holders.

In embodiments of the invention the subject attachment structure comprises an alignment feature for use in aligning the subject attachment structure with an anatomical structure and/or a marking on the subject.

In embodiments of the invention the anatomical structure is a vertebrae.

In embodiments of the invention the marking on the subject is an incision mark, a line of sutures or a scar.

In embodiments of the invention the subject attachment structure is provided with at least one window.

In embodiments of the invention measurement markings are provided adjacent to the track.

In embodiments of the invention the subject attachment structure is configured to substantially contour to the torso.

In embodiments of the invention at least an area of the subject attachment portion is made of a flexible material to enable the subject attachment structure to conform to the body.

In embodiments of the invention the area of the subject attachment structure, which in use aligns with the vertebral column, is made of a substantially flexible material.

In embodiments of the invention the area of the subject attachment structure which aligns with the vertebral column acts as a hinge.

In embodiments of the invention a belt attachment region is provided on at least two opposing sides of the subject attachment structure.

In embodiments of the invention the belt attachment region comprises a slot through which a portion of a belt is passable.

According to a further aspect of the invention there is provided a method of accelerating bone formation in a subject, the method comprising the steps of;
(i) providing an ultrasound delivery system comprising a subject attachment structure having a subject-facing surface which is configured to substantially contour to a region of the subject to which ultrasound is to be delivered, the subject attachment structure comprising;
   a pair of ultrasound transducer holders located on the subject-facing surface, each holder capable of receiving an ultrasound transducer,
   a belt attachment region;
(ii) introducing a ultrasound transducer into each transducer holder;
(iii) placing the subject-facing surface of the subject attachment structure against the treatment site;
(iv) adjusting the position of at least one of the transducer holders in at least one of the medial/lateral, or the inferior/superior or the anterior/posterior position relative to the treatment site;
(v) introducing a belt into the belt attachment region and securing the subject attachment structure at the treatment site.

In embodiments of the invention the angular position of at least one transducer holder can be altered relative to the treatment site.

In embodiments of the invention the method is used for accelerating bone formation in spinal indications.

In embodiments of the invention the method is used to obtain partial or complete vertebral fusion. The method is used in spondylodesis or spondylosyndesis.

In embodiments of the invention the pair of transducer holders are positioned such that a transducer holder is located on either side of a vertebrae.

In embodiments of the invention the subject attachment structure is placed at the iliac crest.

In embodiments of the invention ultrasound is applied to the treatment site on a daily basis.

In embodiments of the invention the ultrasound is applied for a period of from about 10 to about 30 minutes

In embodiments of the invention the subject is a mammal.

In embodiments of the invention the mammal is a human.

In embodiments of the invention mammal is a non-human mammal.

According to a further aspect of the invention, there is provided an apparatus for accelerating spinal fusion. The apparatus comprises a treatment head module housing and a belt. The treatment head module housing has a frame with adjustment rails, at least two yoke attached to the adjustment rails, a plunger movably attached to each of the yoke, and a transducer holder attached to each plunger. The belt may be connected to the treatment head module housing.

According to a further aspect of the invention, there is provided a method of medical treatment. The method comprises performing spinal fusion on a patient's spine; fitting a treatment head module housing to the patient; adjusting a position of at least one of the transducer holders; and treating a spine fusion area with ultrasound on a regular basis for a period of time.

According to a further aspect of the invention there is provided an ultrasound delivery system or a method as substantially herein described with reference to the accompanying Figures.

Further areas of applicability of the invention will become apparent from the detailed description provided hereinafter. It should be understood that the detailed description and specific examples, while indicating the particular embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is a front view of a treatment head module housing in a first embodiment.

FIG. 2 is a back view of the embodiment shown in FIG. 1.

FIG. 3 is a front perspective view of a treatment head module housing in a second embodiment.

FIG. 4 is a front perspective view of the second embodiment illustrated with a belt attached.

FIG. 5 is a front view of a transducer holder in a first embodiment.

FIG. 6 is a back view of the embodiment shown in FIG. 5.

FIG. 7 illustrates variations of the transducer holder shown in FIG. 5.

FIG. 8 is a top perspective view of a transducer holder in a second embodiment.

FIG. 9 is a top perspective view of a transducer holder in a third embodiment.

FIG. 10 is a top perspective view of a transducer holder in a fourth embodiment.

FIG. 11 is a back perspective view of a treatment head module housing in a third embodiment.

FIG. 12 is a front perspective view of the embodiment shown in FIG. 11.

FIG. 13 is a detailed perspective view of a yoke in a first embodiment.

FIG. 14 is an exploded view of the embodiment shown in FIG. 13.

FIG. 15 is a rear perspective view of the embodiment shown in FIG. 14.

FIG. 16 is a back perspective view of a treatment head module housing in a fourth embodiment.

FIG. 17 is a perspective side view of a transducer holder in a fifth embodiment.

FIG. 18 is a side view of the embodiment shown in FIG. 17.

FIG. 19 is a perspective side view of a plunger in an alternative embodiment.

FIG. 20 is a back perspective view of a treatment head module housing in a fifth embodiment.

FIG. 21 is a back perspective view of a treatment head module housing in a sixth embodiment.

FIG. 22 is a back perspective view of a treatment head module housing in a seventh embodiment.

FIG. 23 is a back perspective view of a treatment head module housing in an eighth embodiment.

FIG. 24 is a detailed perspective view of a yoke in a second embodiment.

FIG. 25 is a back perspective view of a treatment head module housing in a ninth embodiment.

FIG. 26 is a detailed perspective view of a yoke in a third embodiment.

FIG. 27 is a back perspective view of a treatment head module housing in a tenth embodiment.

FIG. 28 is a detailed perspective view of a yoke in a fourth embodiment.

FIG. 29 is a back perspective view of a treatment head module housing in an eleventh embodiment.

FIG. 30 is a detailed perspective view of a yoke in a fifth embodiment.

FIG. 31 is a back perspective view of a treatment head module housing in a twelfth embodiment.

FIG. 32 is a back perspective view of a treatment head module housing in a thirteenth embodiment.

FIG. 33 is a back perspective view of a treatment head module housing in a fourteenth embodiment.

FIG. 34 is a back perspective view of a treatment head module housing in a fifteenth embodiment.

FIG. 35 is a back perspective view of a frame of the embodiment shown in FIG. 34.

FIG. 36 is a perspective back view of a transducer holder in a sixth embodiment.

FIG. 37 is a perspective front view of a transducer holder in a seventh embodiment.

FIG. 38 is perspective back view of the embodiment shown in FIG. 37.

FIG. 39 is a perspective front view of a transducer holder in an eighth embodiment.

FIG. 40 is a back perspective view of a treatment head module housing in a sixteenth embodiment.

FIG. 41 is a back perspective view of a treatment head module housing in a seventeenth embodiment.

FIG. 42 is a back perspective view of a treatment head module housing in an eighteenth embodiment.

FIG. 43 is a back perspective view of a treatment head module housing in a nineteenth embodiment.

FIG. 44 is a detailed perspective view a transducer holder in a ninth embodiment.

FIG. 45 is a rear perspective view of the embodiment shown in FIG. 44.

FIG. 46 is a back perspective view of a treatment head module housing in a twentieth embodiment.

FIG. 47 is a detailed perspective view a transducer holder in a tenth embodiment.

FIG. 48 is a back view of a treatment head module housing in a twenty-first embodiment.

FIG. 49 is a perspective view of the embodiment shown in FIG. 48.

FIG. 50 illustrates variations of the transducer holder shown in FIG. 49

FIG. 51 is a sectional view of a yoke in a sixth embodiment.

FIG. 52 is a sectional view of a yoke in a seventh embodiment.

FIG. 53 is a sectional view of a yoke in an eighth embodiment.

FIG. 54 is a sectional view of a yoke in a ninth embodiment.

FIG. 55 is a sectional view of a yoke in a tenth embodiment.

FIG. 56 is a sectional view of a yoke in an eleventh embodiment.

FIG. 57 is a sectional view of a yoke in a twelfth embodiment.

FIG. 58 is a sectional view of a yoke in a thirteenth embodiment.

FIG. 59 is a sectional view of a yoke in a fourteenth embodiment.

FIG. 60 is a back view of a treatment head module housing in a twenty-second embodiment.

FIG. 61 is a detailed front perspective view of the embodiment shown in FIG. 60.

FIG. 62 is a detailed rear perspective view of the embodiment shown in FIG. 60.

FIG. 63 is a back view of a treatment head module housing in a twenty-third embodiment.

FIG. 64 is a front perspective view of the embodiment shown in FIG. 63.

FIG. 65 is a partially exploded, rear perspective view of the embodiment shown in FIG. 63.

FIG. 66 is a detailed perspective view of a treatment head module housing in a twenty-fourth embodiment.

FIG. 67 is a detailed perspective view a transducer holder in an eleventh embodiment.

FIG. 68 is a detailed top perspective view a transducer holder in a twelfth embodiment.

FIG. 69 is a detailed bottom perspective view of the embodiment shown in FIG. 68.

FIG. 70 is a front perspective view of a transducer holder in a thirteenth embodiment.

FIG. 71 is a front perspective view of the transducer holder shown in FIG. 70 with the cover removed.

FIG. 72 is a detailed perspective view of a yoke in a fifteenth embodiment.

FIG. 73 is a first belt configuration.

FIG. 74 is a second belt configuration.

FIG. 75 is third belt configuration.

FIG. 76 is a fourth belt configuration.

FIG. 77 is a fifth belt configuration.

FIG. 78 illustrates transducer signals of the prior art.

FIG. 79 illustrates transducer signals of one particular embodiment.

FIG. 80 illustrates a system for applying transducer signals.

FIG. 81 is a block diagram of the controller shown in FIG. 80.

FIG. 82 is a flowchart illustrating a method of ultrasound application.

FIG. 83 is an instrument for selecting transducer placement.

### DETAILED DESCRIPTION OF THE EMBODIMENTS

The following description of the depicted embodiment(s) is merely exemplary in nature and is in no way intended to limit the invention, its application, or uses.

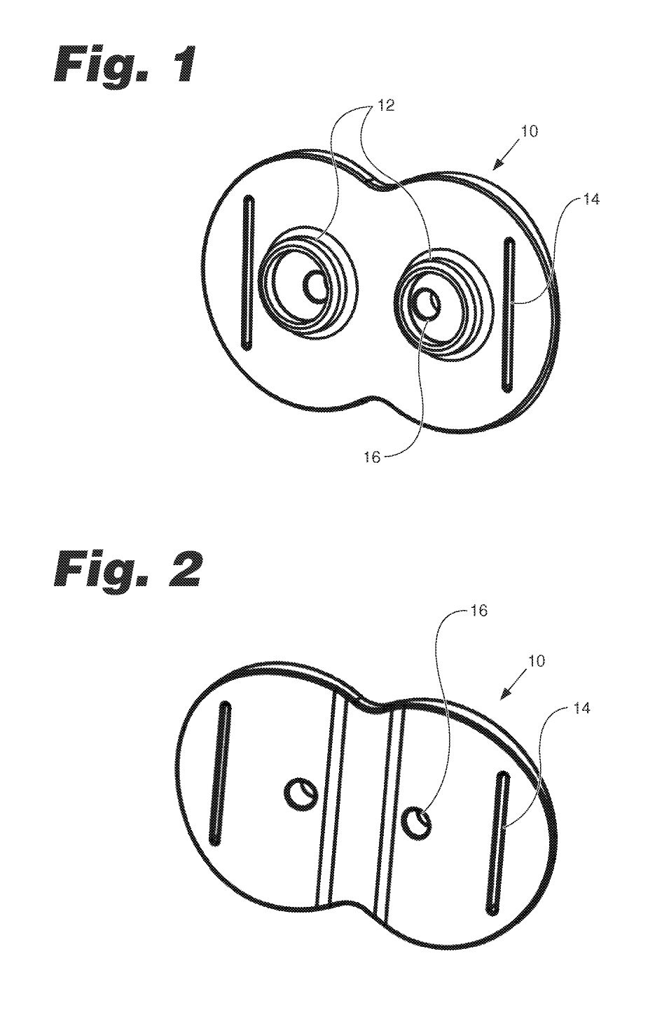

FIGS. 1 and 2 illustrate a treatment head module housing 10. The treatment head module housing 10 includes a pair of transducer holders 12 and a pair of belt insert openings 14. Each transducer holder 12 is adapted to hold a transducer (not shown) and includes a wire opening 16 to receive a wire (not shown) attached to the transducer. A belt (not shown) may be attached to one or both of the belt insert openings 14.

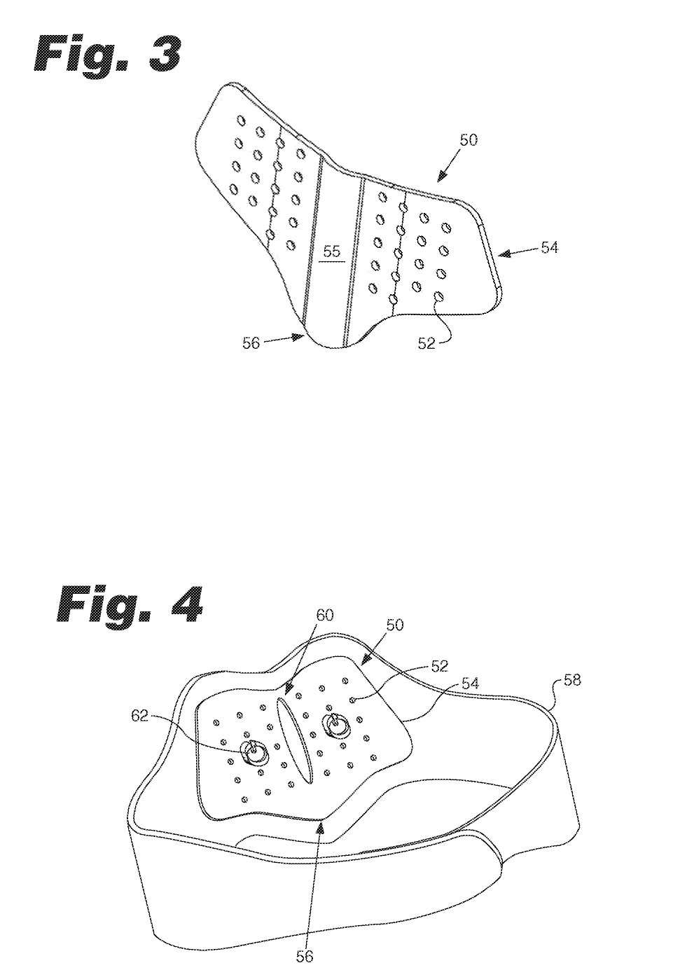

FIGS. 3 and 4 illustrate a second embodiment of the treatment head module housing 50. The treatment head module housing 50 includes positioning holes 52, belt attachment members 54, and an alignment portion 56. A belt 58 is attached to the treatment head module housing 50 at the belt attachment members 54. Transducer holders 62 may be selectively positioned in the positioning holes 52. In the depicted embodiment, the positioning holes are arranged in a grid-like fashion. In some embodiments, the treatment head module housing 50 includes an incision window 60. In some embodiments, the treatment head module housing 50 may include a flex portion 55. The flex portion 55 is more flexible than the belt attachment members 54 and allows the treatment head module housing 50 to conform to a patient's back.

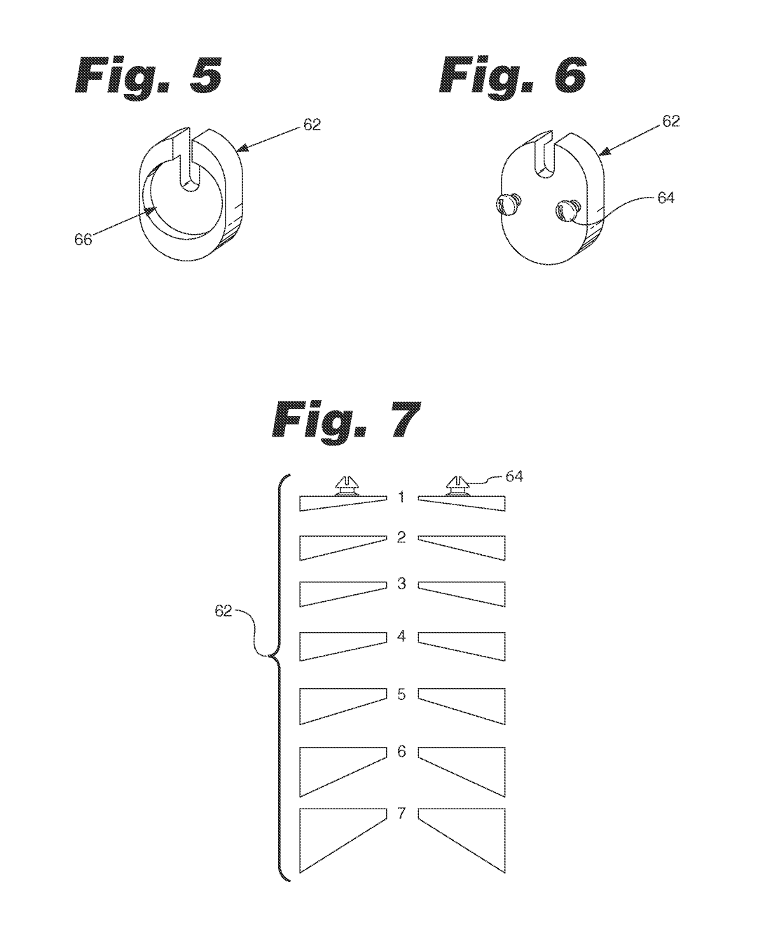

FIGS. 5-7 illustrate the transducer holder 62. The transducer holder 62 includes a dished portion or cup 66. The cup 66 is adapted to receive a transducer (not shown). One or more fasteners 64 may be used to attach the transducer holder 62 to the treatment head module housing 50. As best seen in FIG. 7, the transducer holder 62 may be shaped to transmit ultrasound signals from the transducer towards a patient's spine. The transducer holder 62 may be angled relative to the treatment head module housing 50. As illustrated in FIG. 7, the angle may range from about 1 degree to about 50 degrees, and more particularly from about 1 degree to about 35 degrees.

FIG. 8 is a top perspective view of a transducer holder in a second embodiment. The transducer holder 70 has a backing layer 72, a base 74, and an adhesive layer 76. The backing layer 72 is removed to reveal the adhesive layer 76. A transducer 78 may be attached to the base 74 via the adhesive layer 76.

FIG. 9 is a top perspective view of a transducer holder in a third embodiment. The transducer holder 80 includes a first layer 82 and a second layer 84. The second layer 84 may include a dish portion or cup 86 adapted to receive a transducer.

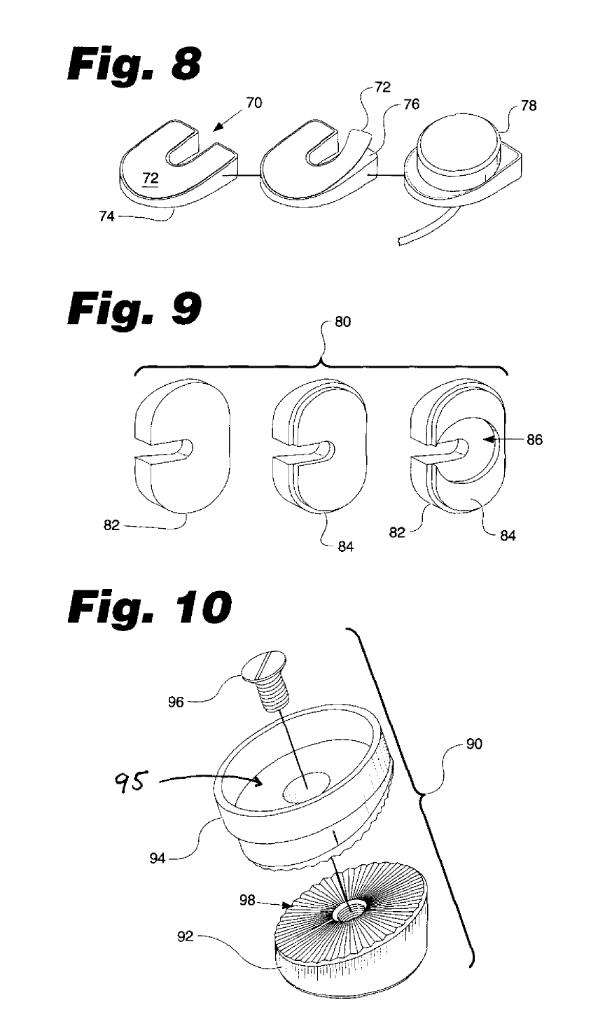

FIG. 10 is a top perspective view of a transducer holder in a fourth embodiment. The transducer holder 90 includes a lower portion 92, an upper portion 94, and a fastener 96 to connect the upper and lower portions 92, 94. The upper portion 94 includes a dish portion or cup 95 adapted to receive the transducer. The upper and lower portions 92, 94 have a complementary shape and complementary teeth 98. The upper portion 94 may be rotated relative to the lower portion 92 to achieve a desired angle of the transducer.

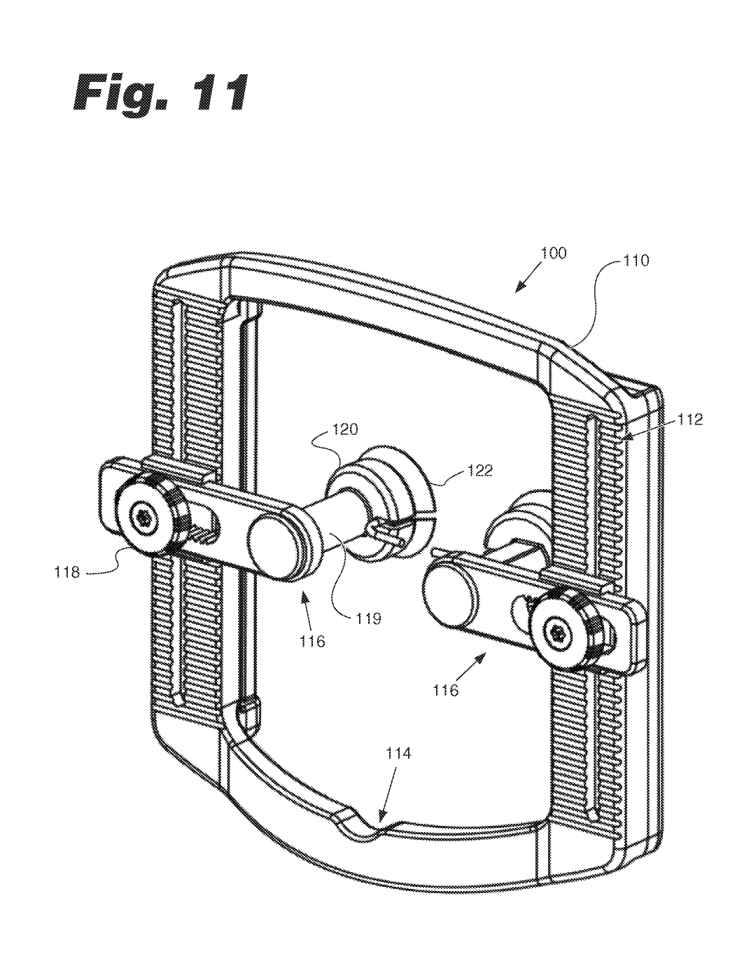

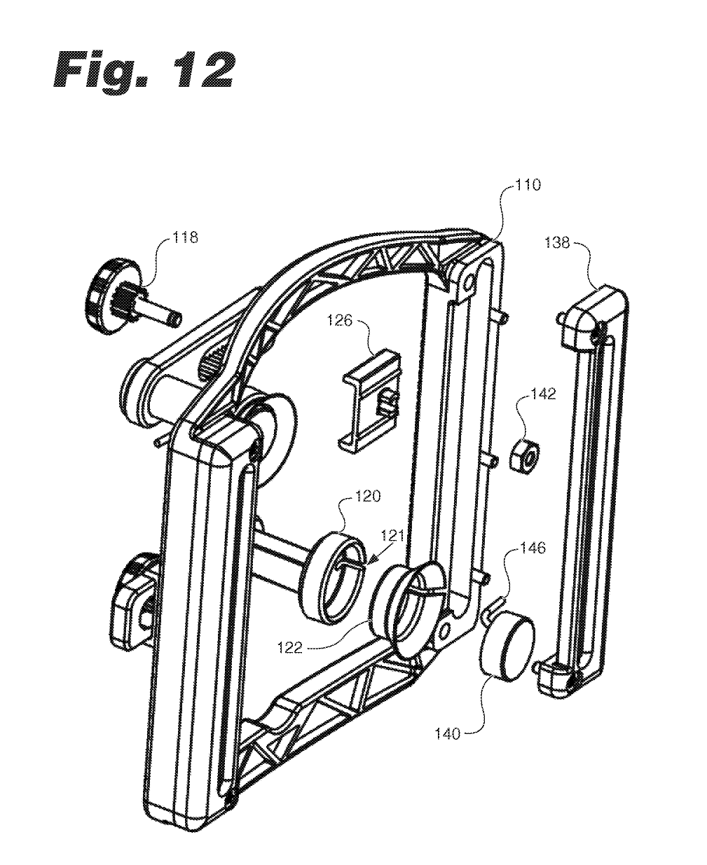

FIGS. 11-15 illustrate a treatment head module housing 100. The treatment head module housing 100 includes a frame 110 and adjustment rails 112. In some embodiments, the treatment head module housing 100 includes an alignment feature 114. A pair of yoke 116 is removably attached to the frame 110. In some embodiments, one or more of the yokes may be permanently affixed to the frame 110. A horizontal adjustment knob 118 is used to adjust the yoke 116 in a medial/lateral direction. As best seen in FIG. 14, the horizontal adjustment knob 118 may include a first fastener 124. The first fastener 124 may be connected to a second fastener 142, such as a nut, to lock the horizontal adjustment knob. A transducer holder 120 is connected to the yoke 116 through the use of a plunger 119. In the depicted embodiment the plunger is D-shaped. In other embodiments, the plunger 119 may be cylindrical. Those having ordinary skill in the art would understand that other shapes, such as triangular or square, may be used.

The transducer holder 120 receives a transducer 140. In some embodiments, the transducer holder 120 includes a slot 121 to receive a wire 146 of the transducer 140. A gel retaining cup 122 may be placed in-between the transducer 140 and the transducer holder 120. In the depicted embodiment, the gel-retaining cup is generally frusto-conical. The gel retaining cup 122 may be used to retain a gel. In some embodiments, gel is placed in the retaining cup 122, the cup is placed against the patient's skin, and the cone opens up upon placement against the patient's skin. The gel retaining cup 122 reduces the possibility of gel spillage.

In some embodiments, the transducer holder 120 may be angled relative to the yoke 116. This angle may be in the range from about zero degrees to about fifty degrees. In the depicted embodiment, the transducer holder 120 is at an angle of about five degrees.

The frame 110 includes a pair of belt attachment members 138. The belt attachment members 138 are used to attach a belt (not shown) to the frame 110. The belt attachment members 138 may be integral with the frame 110, but in the depicted embodiment the pair of belt attachment members 138 are removably attached to main section of the frame 110.

As best seen in FIG. 14, an alignment member 126 is placed in-between the yoke 116 and the rail 112. The rail 112 may include one or more grooves or channels 113. The alignment member 126 may include one or more rail boss 136. The rail boss 136 is complementary to the channel 113 and is adapted to locate the alignment member 126 in a proximal/distal direction. Of course, those skilled in the art would understand that the channel 113 and the rail boss 136 may reversed in position. In some embodiments, the alignment member 126 includes one or more flanges 127 to prevent rotation of the yoke 116. In some embodiments, the rail 112 includes a rail opening 132, and the alignment member 126 includes a corresponding alignment guide 134. The alignment guide 134 extends within the rail opening 132 to locate the alignment member 126 in a medial/lateral direction. The alignment guide 134 may have certain geometric features, such as generally parallel sides, that complement the rail opening 132 to substantially prevent rotation of the alignment member 126. While the rail opening 132 is illustrated as one continuous slot, those of ordinary skill in the art would understand that the rail opening 132 could equally be a series of holes. In some embodiments, the frame 110 has a generally arcuate shape from top-to-bottom. In some embodiments, the frame 110 has a generally arcuate shape from side-to-side. In yet another embodiment, the frame 110 has a generally arcuate shape from top-to-bottom and from side-to-side.

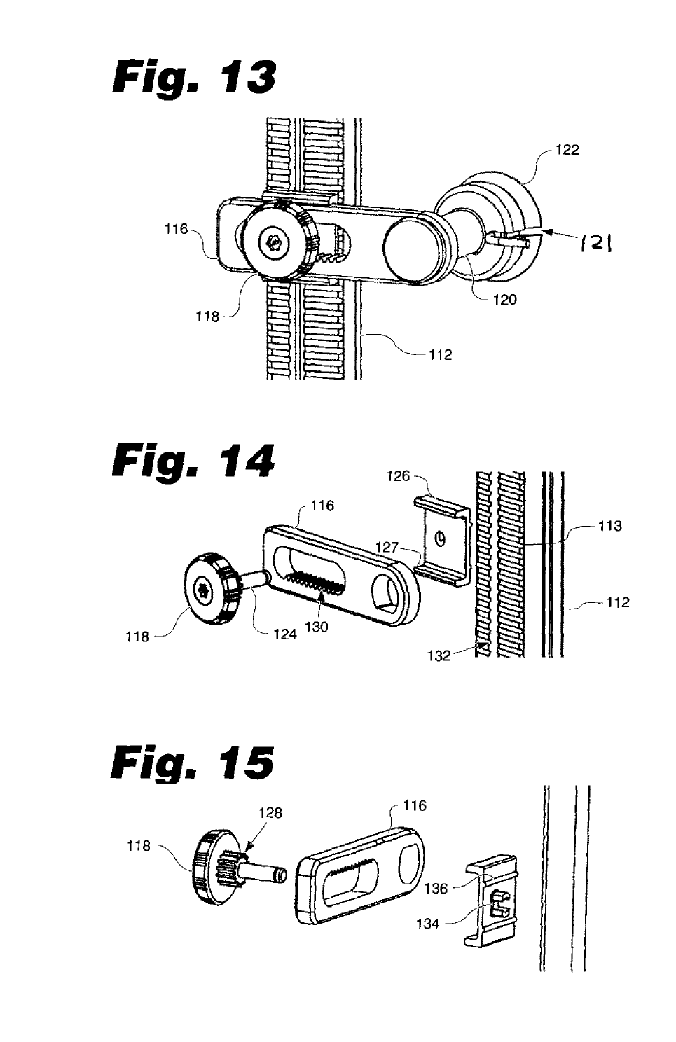

FIGS. 13-15 illustrate one particular embodiment of the yoke 116. In the depicted embodiment, the yoke 116 includes gear teeth 130, and the horizontal adjustment knob includes a corresponding gear member 128. The teeth of the gear member 128 are adapted to mesh with the gear teeth 130 such that as the gear member 128 is rotated, the yoke 116 is moved in a medial/lateral direction. In the depicted embodiment, the yokes 116 may be placed a minimum of about 45 mm apart or a maximum of about 120 mm apart.

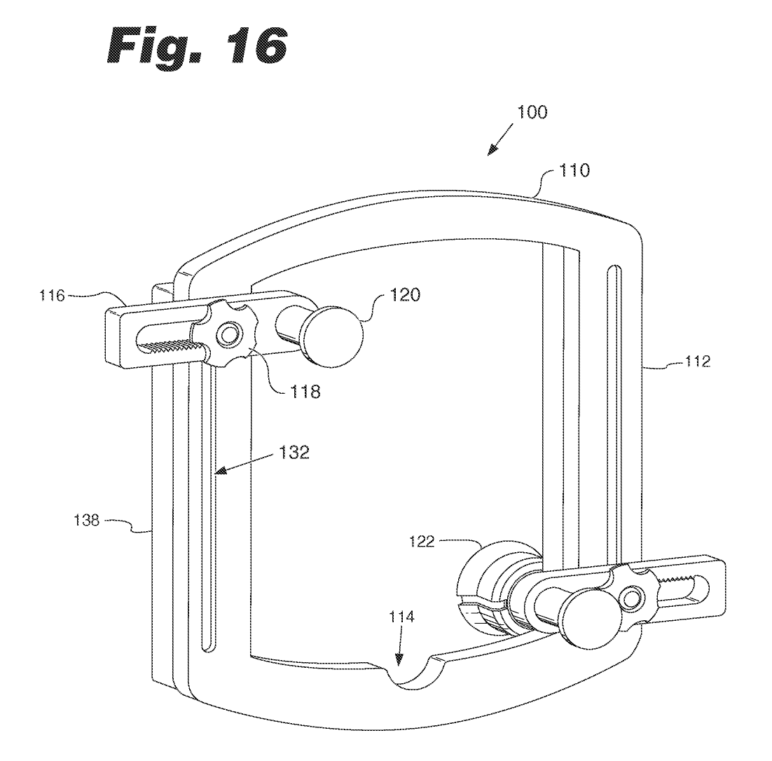

FIG. 16 is an alternative version of the treatment head module housing 100. In the embodiment depicted in FIG. 16, the treatment head module housing 100 includes the frame 110 and adjustment rails 112. In the embodiment depicted in FIG. 16, the rails do not include grooves or channels but do include one or more rail openings 132. In some embodiments, the treatment head module housing 100 includes the alignment feature 114. The pair of yoke 116 is removably attached to the frame 110. In some embodiments, one or more of the yokes may be permanently affixed to the frame 110. The horizontal adjustment knob 118 is used to adjust the yoke 116 in a medial/lateral direction. The transducer holder 120 is connected to the yoke 116. The transducer holder 120 receives the transducer 140. The gel retaining cup 122 may be placed in-between the transducer 140 and the transducer holder 120. In some embodiments, the transducer holder 120 may be angled relative to the yoke 116. This angle may be in the range from about zero degrees to about fifty degrees. In the depicted embodiment, the transducer holder 120 is at an angle of about five degrees. In some embodiments, the frame 110 has a generally arcuate shape from top-to-bottom. In some embodiments, the frame 110 has a generally arcuate shape from side-to-side. In yet another embodiment, the frame 110 has a generally arcuate shape from top-to-bottom and from side-to-side.

The frame 110 includes the pair of belt attachment members 138. The belt attachment members 138 are used to attach a belt (not shown) to the frame 110. The belt attachment members 138 may be integral with the frame 110, but in the depicted embodiment the pair of belt attachment members 138 are removably attached to main section of the frame 110.

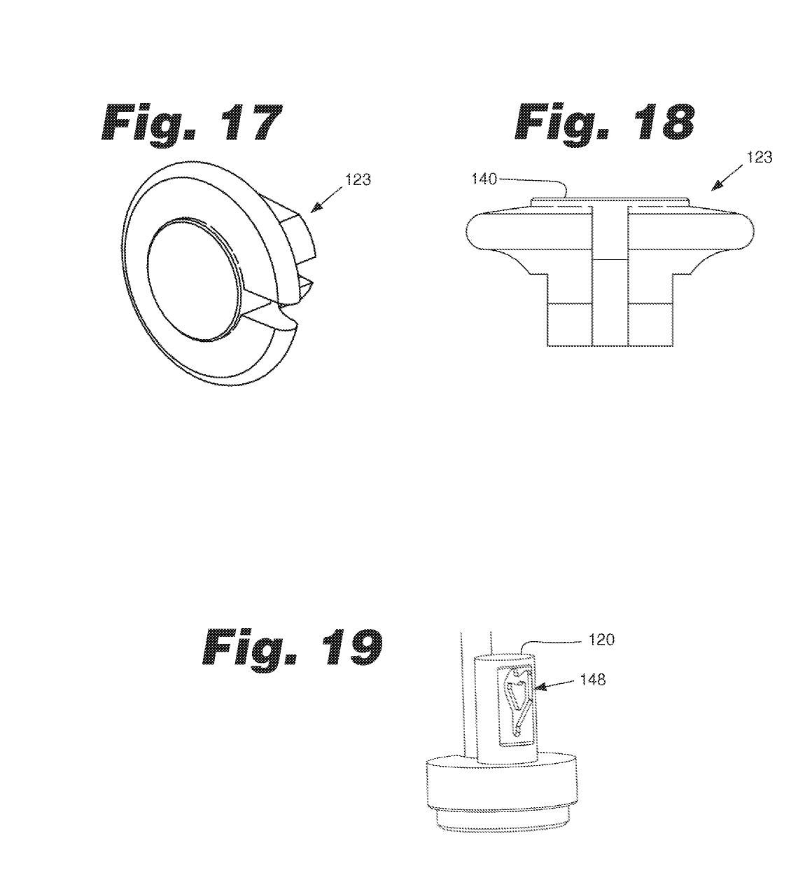

FIGS. 17-18 illustrate a transducer holder in a fifth embodiment. The transducer holder 123 has a generally hemispherical shape and a central pocket that holds the transducer 140.

FIG. 19 is a perspective side view of a plunger in an alternative embodiment. The plunger 120 includes a cam 148. The cam 148 allows for two-position operation, similar to a ball point pen.

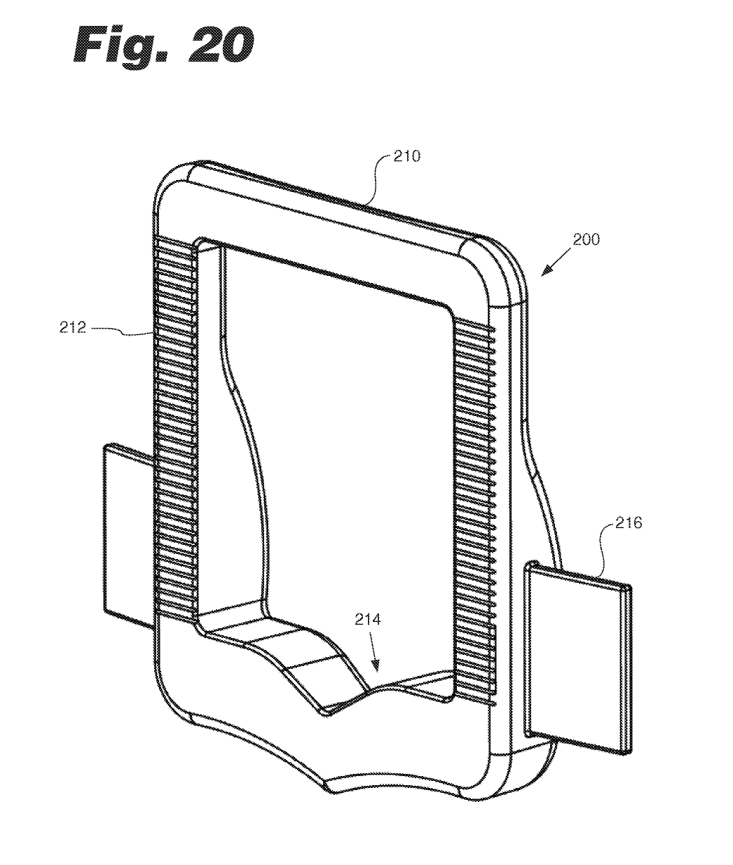

FIG. 20 is a back perspective view of a treatment head module housing in a fifth embodiment. The treatment head module housing 200 includes a frame 210, adjustment rails 212, and belt attachment members 216. In some embodiments, the treatment head module housing 200 also includes an alignment feature 214. In some embodiments, a belt (not shown) may be permanently or removably attached to the belt attachment members 216.

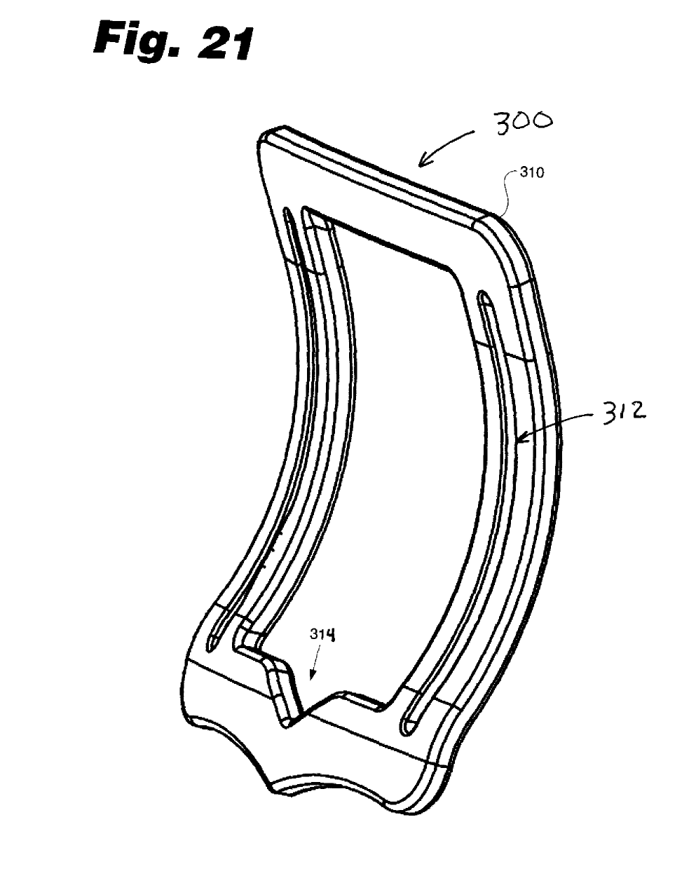

FIG. 21 is a back perspective view of a treatment head module housing in a sixth embodiment. The treatment head module housing 300 includes a frame 310 and adjustment rails 312. In some embodiments, the treatment head module housing 300 also includes an alignment feature 314. In the depicted embodiment, the frame 310 has a generally arcuate shape from top-to-bottom. In some embodiments, the frame 310 has a generally arcuate shape from side-to-side. In yet another embodiment, the frame 310 has a generally arcuate shape from top-to-bottom and from side-to-side.

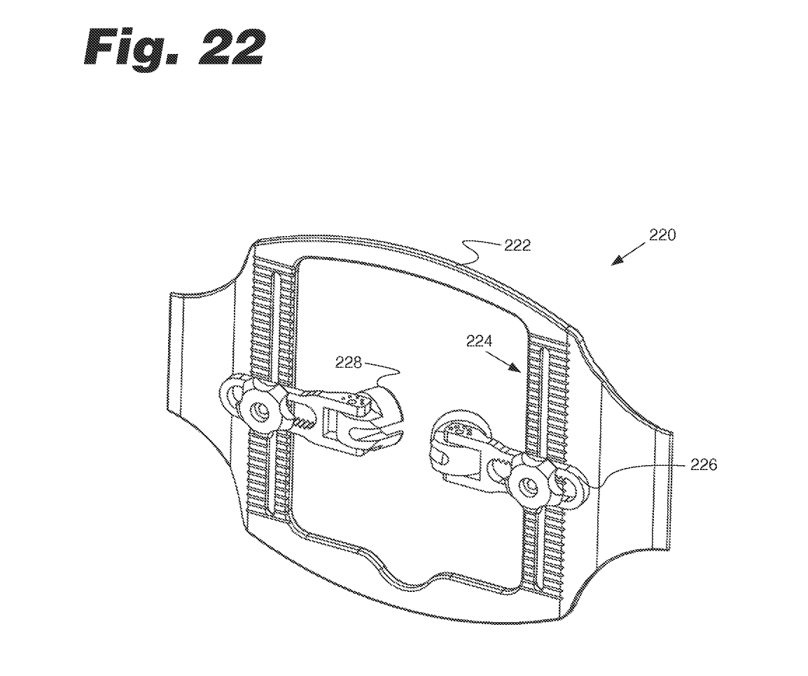

FIG. 22 is a back perspective view of a treatment head module housing in a seventh embodiment. The treatment head module housing 220 includes a frame 222 and may include one or more yokes 226 attached to the frame 222. The frame 222 includes one or more rails 224. A transducer holder 228 may be connected to each yoke 226. In the depicted embodiment, the transducer holder 228 is pivotable relative to the yoke 226. The transducer holder 228 may be angularly adjusted and temporarily locked into place.

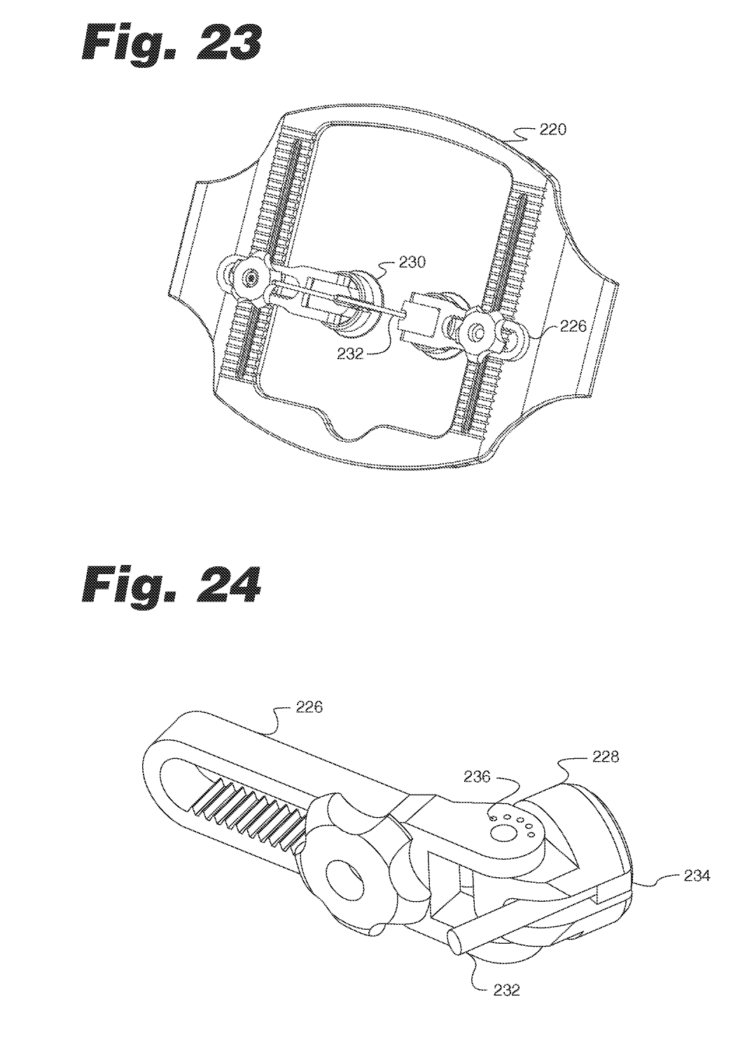

FIG. 23 is a back perspective view of a treatment head module housing in an eighth embodiment. In the depicted embodiment, the yoke 226 further includes a gel retaining cup 230, a transducer (not shown), and a wire or cable 232 extending from the transducer.

FIG. 24 is a detailed perspective view of a yoke in a second embodiment. The yoke 226 includes the transducer holder 228. In the depicted embodiment, a transducer 234 is placed within the transducer holder 228 and a wire or cable 232 extends from the transducer 234. The yoke 226 and the transducer holder 228 have holes 236. A fastener (not shown), such as a removable pin, may be placed in the aligned holes 236 to retain a selected angle adjustment.

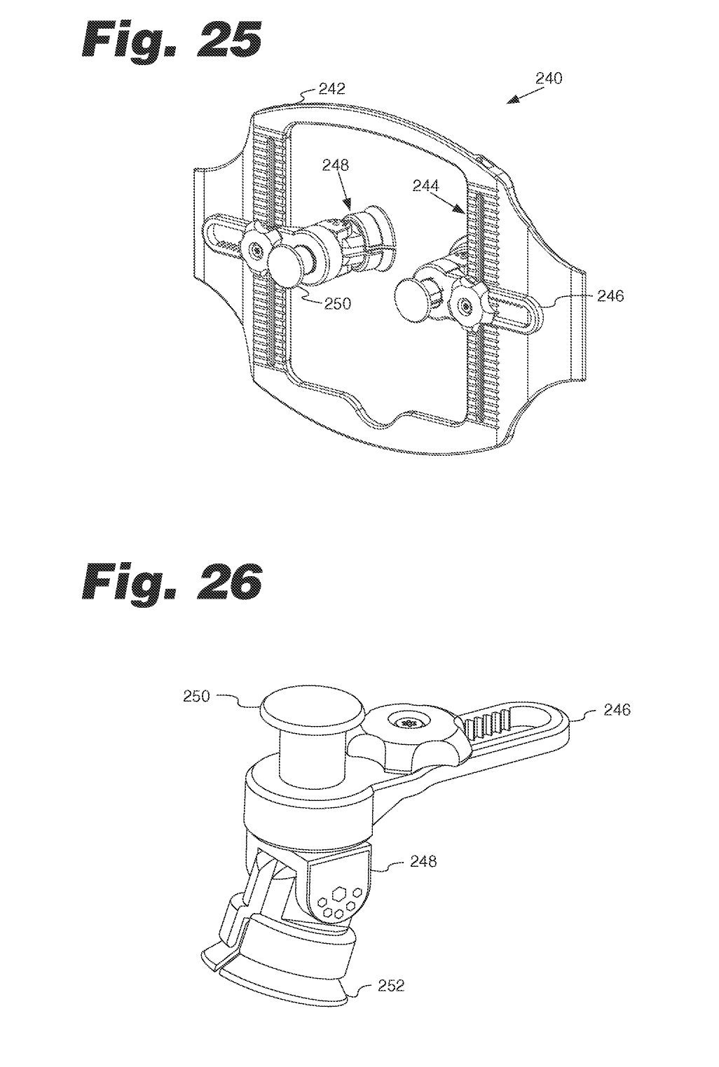

FIG. 25 is a back perspective view of a treatment head module housing in a ninth embodiment. The treatment head module housing 240 includes a frame 242 and one or more yokes 246. The frame 242 includes one or more rails 244. A transducer holder 248 may be connected to the yoke 246. In the depicted embodiment, a plunger 250 connects the transducer holder 248 to the yoke 246 and movement of the plunger 250 changes the anterior/posterior position of the transducer holder 248. A gel retaining cup 252 may be associated with the transducer holder 248. The plunger 250 may be spring biased to make it more likely that the gel retaining cup 252 contacts the patient's skin.

FIG. 26 is a detailed perspective view of a yoke in a third embodiment. In the depicted embodiment, the yoke 246 includes the transducer holder 248, the plunger 250, and the gel retaining cup 252.

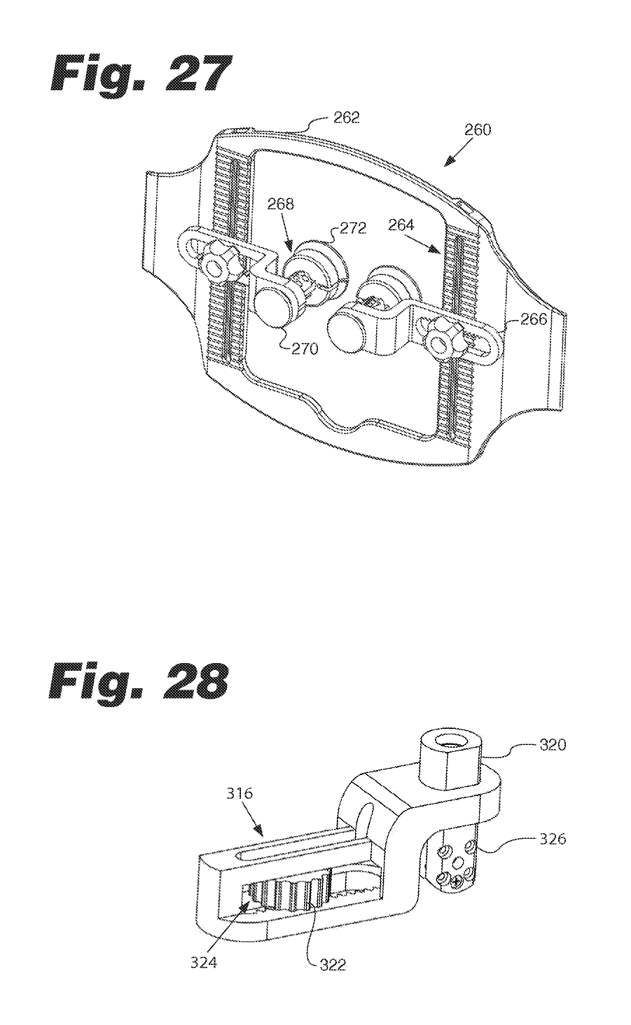

FIG. 27 is a back perspective view of a treatment head module housing in a tenth embodiment. The treatment head module housing 260 includes a frame 262 and one or more yokes 266. The frame 262 includes one or more rails 264. In the depicted embodiment, the yoke 266 is recessed or has an offset portion for longer travel depth adjustment. A transducer holder 268 may be connected to the yoke 266. In the depicted embodiment, a plunger 270 connects the transducer holder 268 to the yoke 266 and movement of the plunger 270 changes the anterior/posterior position of the transducer holder 268. A gel retaining cup 272 may be associated with the transducer holder 268. The plunger 270 may be spring biased to make it more likely that the gel retaining cup 272 contacts the patient's skin.

FIG. 28 is a detailed perspective view of a yoke in a fourth embodiment. The yoke 316 has a captured gear 322. The yoke 316 includes a track 324, and the captured gear 322 is located in the track 324. A plunger 320 may be attached to the yoke 316. The plunger 320 may include holes 326. The holes 326 may be used with a transducer holder and/or a transducer cup (not shown) to achieve a selected angular adjustment of the transducer. In the depicted embodiment, the yoke 316 is recessed or has an offset portion for longer travel depth adjustment. The captured gear 322 may be rotated to move the yoke in a medial/lateral direction.

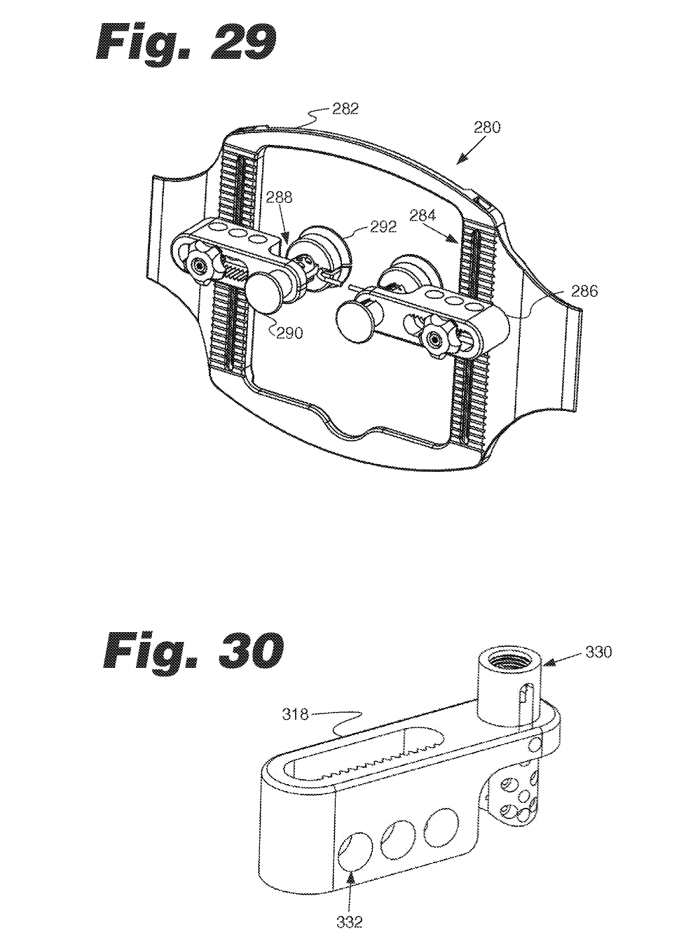

FIG. 29 is a back perspective view of a treatment head module housing in an eleventh embodiment. The treatment head module housing 280 includes a frame 282 and one or more yokes 286. The frame 282 includes one or more rails 284. In the depicted embodiment, the yoke 286 is recessed or has an offset portion for longer travel depth adjustment and also has a thicker portion to provide stability for the yoke. A transducer holder 288 may be connected to the yoke 286. In the depicted embodiment, a plunger 290 connects the transducer holder 288 to the yoke 286 and movement of the plunger 290 changes the anterior/posterior position of the transducer holder 288. A gel retaining cup 292 may be associated with the transducer holder 288. The plunger 290 may be spring biased to make it more likely that the gel retaining cup 292 contacts the patient's skin.

FIG. 30 is a detailed perspective view of a yoke in a fifth embodiment. The yoke 318 includes one or more windows 332 to reduce weight. Although windows 332 are illustrated as circular, other geometric shapes may be used. A plunger 330 may be attached to the yoke 318.

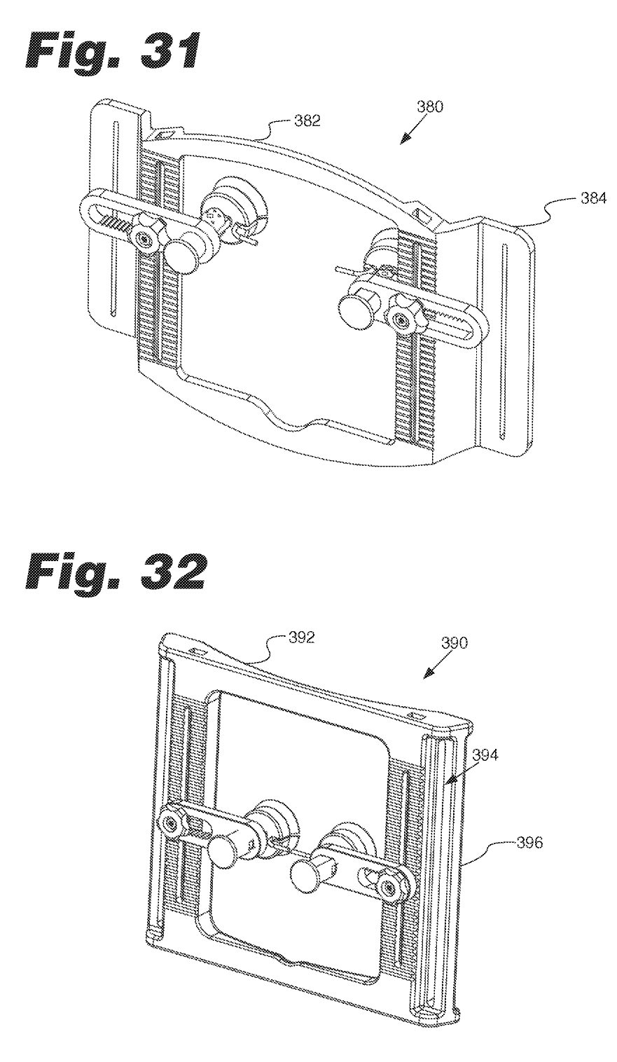

FIG. 31 is a back perspective view of a treatment head module housing in a twelfth embodiment. The treatment head module housing 380 includes a frame 382. The frame 382 may include a wing-shaped belt attachment structure 384. The wing-shaped belt attachment structure 384 may extend the whole length from top-to-bottom or cover only a portion thereof.

FIG. 32 is a back perspective view of a treatment head module housing in a thirteenth embodiment. The treatment head module housing 390 includes a frame 392. The frame 392 may include a cutout 394 proximate to or adjacent to a belt attachment structure 396 for the reduction of weight. The cutout 394 may extend the whole length from top-to-bottom or cover only a portion thereof.

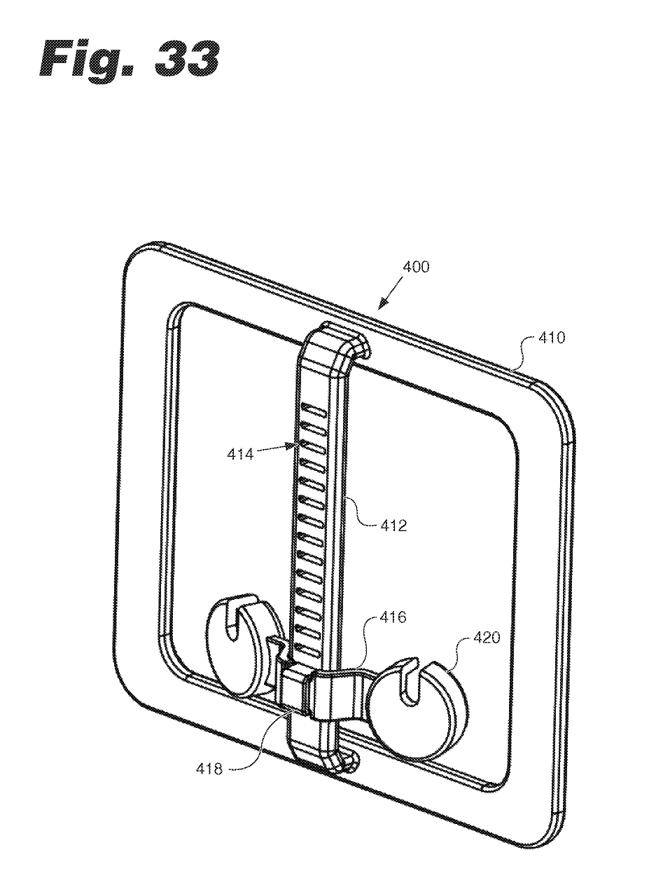

FIG. 33 is a back perspective view of a treatment head module housing in a fourteenth embodiment. The treatment head module housing 400 includes a frame 410 and a rail 412. In the depicted embodiment, the rail 412 is offset from a generally planar face of the frame 410 but the rail 412 could equally be substantially co-planar. The rail 412 may include one or more openings 414. A wrench 416 is removably attached to the rail 412. In some embodiments, a clip 418 is used to hold the wrench in place relative to the rail 412. The clip 418 snaps into the openings 414. The wrench 416 may be adjusted proximally or distally and held at the selected location via the clip 418. The wrench 416 may have one or more transducer holders 420. The wrench may be shaped to angle the transducer holder relative to the frame 410 or relative to one another. In the depicted embodiment, the wrench 416 includes two transducer holders 420 which are separated a fixed equal distance from the rail 412. However, those of ordinary skill in the art would understand that the transducer holders 420 may be separated a fixed unequal distance from the rail 412.

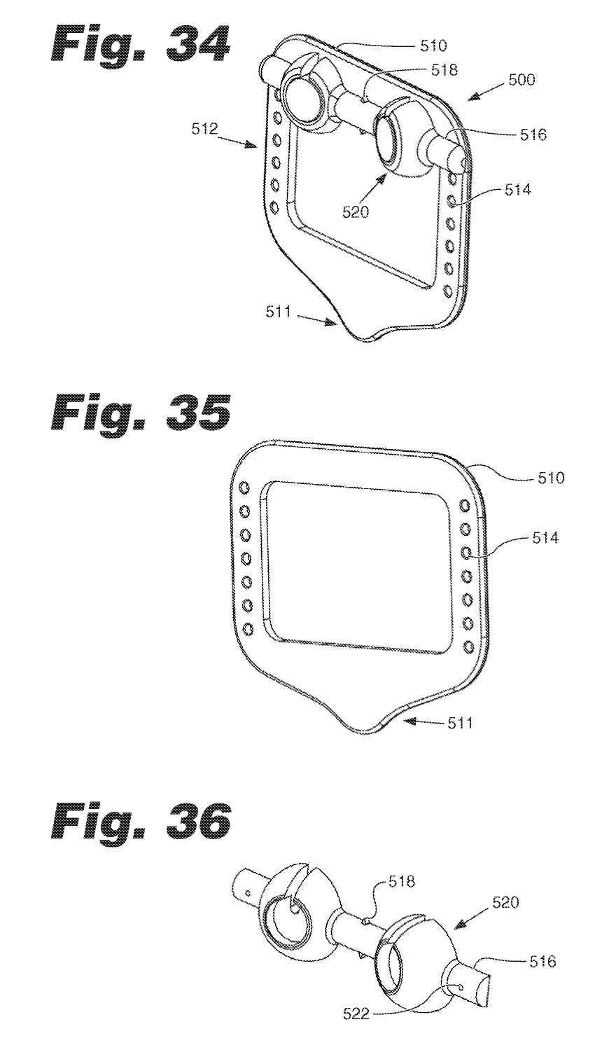

FIGS. 34-36 illustrate a treatment head module housing in a fifteenth embodiment. The treatment head module housing 500 includes a frame 510 and rails 512. In some embodiments, the frame 510 includes an alignment feature 511. The rail 512 may include one or more openings 514. A wrench 516 is removably attached to the rails 512. The wrench 516 may be adjusted proximally or distally and held at the selected location via the openings 514. The wrench 516 may have one or more transducer holders 520. The wrench may be shaped to angle the transducer holder relative to the frame 510. In the depicted embodiment, the wrench 516 includes two transducer holders 520 which are equally spaced relative to the rails 512. However, those of ordinary skill in the art would understand that the transducer holders 520 may be unequally spaced from the rails 512. In some embodiments, the wrench includes one or more alignment features 518. The alignment feature 518 may be aligned with anatomy or scar tissue. FIG. 35 is a back perspective view of a frame of the embodiment shown in FIG. 34. In the embodiment depicted in FIG. 36, the wrench 516 includes a pin 522 for connection to the opening 514. The pin 522 may be removably attached or an integral component.

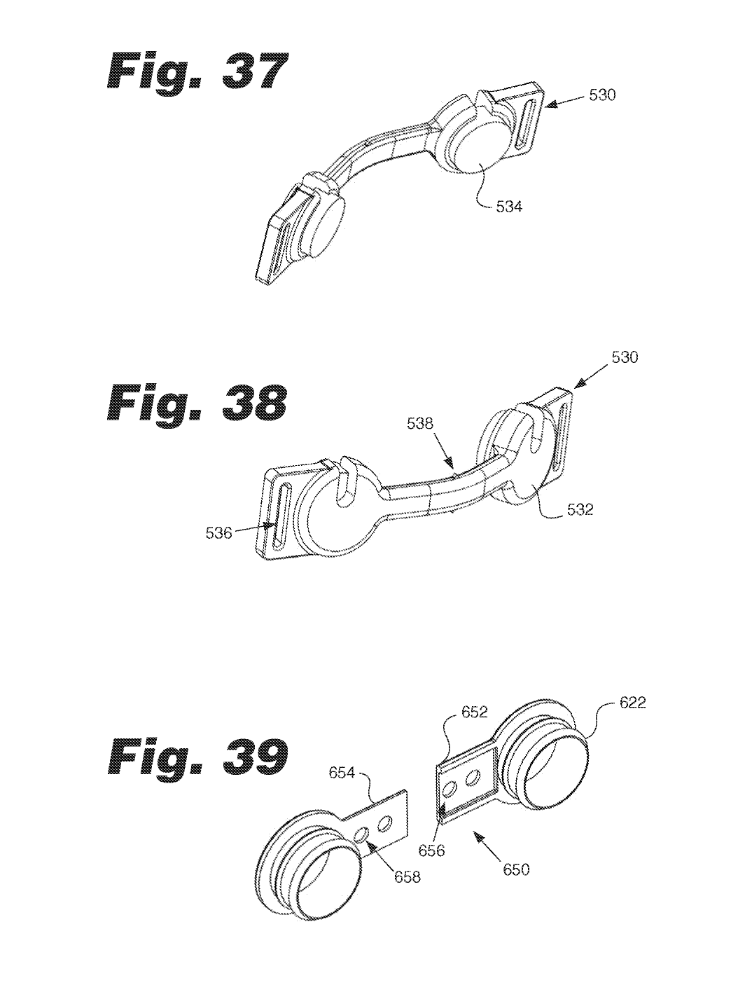

FIGS. 37-38 illustrate a transducer holder in a seventh embodiment. The transducer holder is in the form of a wrench 530. The wrench 530 includes one or more transducer cups 532, each of which are adapted to hold a transducer 534. The wrench may include one or more slots 536. In some embodiments, the wrench 530 includes an alignment feature 538.

FIG. 39 is a perspective front view of a transducer holder in an eighth embodiment. The transducer holder is in the form of a wrench 650. The wrench 650 has a first portion 652 and a second portion 654. Each portion 652, 654 has a transducer cup 622. The first portion 652 has a channel 656 that receives a tab 658 of the second portion 654. Each portion 652, 654 may include one or more holes to connect the wrench 650 to a frame (not shown).

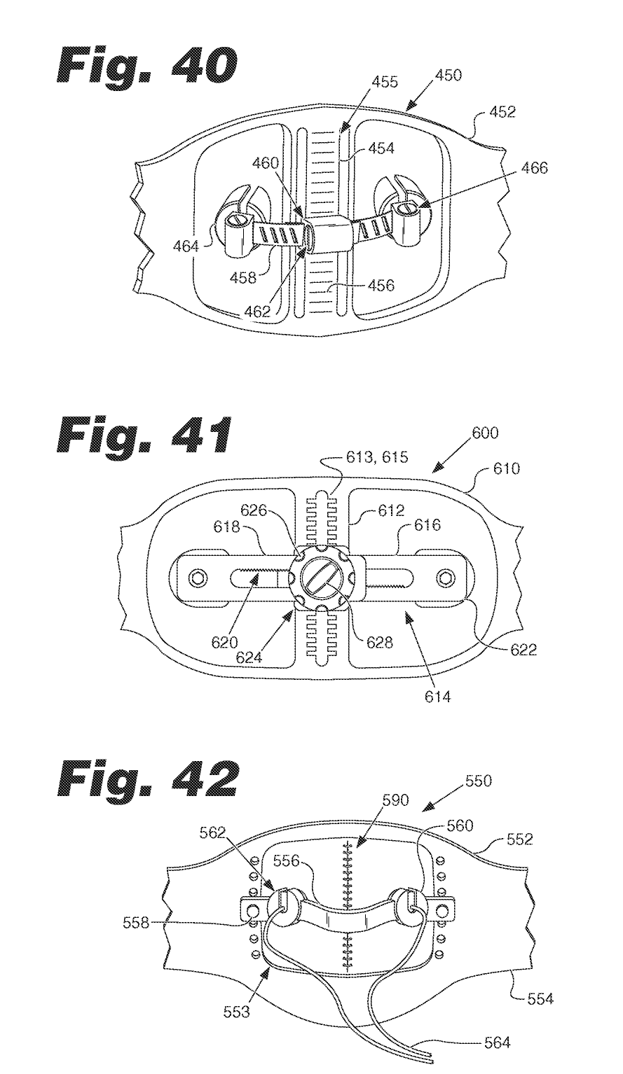

FIG. 40 is a back perspective view of a treatment head module housing in a sixteenth embodiment. The treatment head module housing 450 includes a frame 452 and a rail 454. In the depicted embodiment, the rail 454 is substantially planar with a generally planar face of the frame 452 but the rail 454 could equally be offset. A wrench 458 is removably attached to the rail 454. The rail 454 may include one or more slots 455 and markings 456. As examples, the markings 456 may be detents or protrusions to aid in measuring position of the wrench 458. The wrench 458 may include tabs (not shown) that extend into the slots 455. The wrench 458 may be adjusted proximally or distally and held at the selected location via friction between the tabs and the rail 454. The wrench 458 may have one or more transducer holders 464. The wrench 458 may be shaped to angle the transducer holder relative to the frame 452. In the depicted embodiment, the wrench 458 includes two transducer holders 464 which are separated a fixed equal distance from the rail 454. However, those of ordinary skill in the art would understand that the transducer holders 464 may be separated a fixed unequal distance from the rail 454. In some embodiments, the wrench 458 includes a screw mechanism or a worm mechanism 462 to adjust the transducer holders 464 toward or away from the rail 454. The screw mechanism 462 is similar in operation as to that of a screw and band-type hose clamp. In some embodiments, the wrench 458 or the transducer holder 464 may include a locking mechanism 466 such that the transducer holder 464 is held at a fixed angle after adjustment.

FIG. 41 is a back perspective view of a treatment head module housing in a seventeenth embodiment. The treatment head module housing 600 includes a frame 610 and a rail 612. In the depicted embodiment, the rail 612 is generally co-planar with a generally planar face of the frame 610 but the rail 612 could equally be offset. The rail 612 may include one or more openings 613 with horizontal slots 615. A rack-and-pinion 614 is removably attached to the rail 612. The rack-and-pinion 614 may include a first rack 616, a second rack 618, and a knob assembly 624. Each rack 616, 618 may include teeth 620. The knob assembly 624 includes an outer dial 626 and an inner dial 628. The outer dial 626 includes teeth (not shown) to engage the teeth 620. The outer dial 626 may be rotated to move racks 616, 618 toward or away from the rail 612. The inner dial 628 may include a fastener portion (not shown) adapted to engage a friction plate (not shown). The friction plate may engage the horizontal slots 615. The rack-and-pinion 614 may be adjusted proximally or distally and held at the selected location via the inner dial 628 and the friction plate. The rack-and-pinion 614 may have one or more transducer holders 622. The rack-and-pinion 614 may be shaped to angle the transducer holder relative to the frame 610. In the depicted embodiment, the rack-and-pinion 614 includes two transducer holders 622 which are separated a fixed equal distance from the rail 612. However, those of ordinary skill in the art would understand that the transducer holders 622 may be separated a fixed unequal distance from the rail 612.

FIG. 42 is a back perspective view of a treatment head module housing in an eighteenth embodiment.

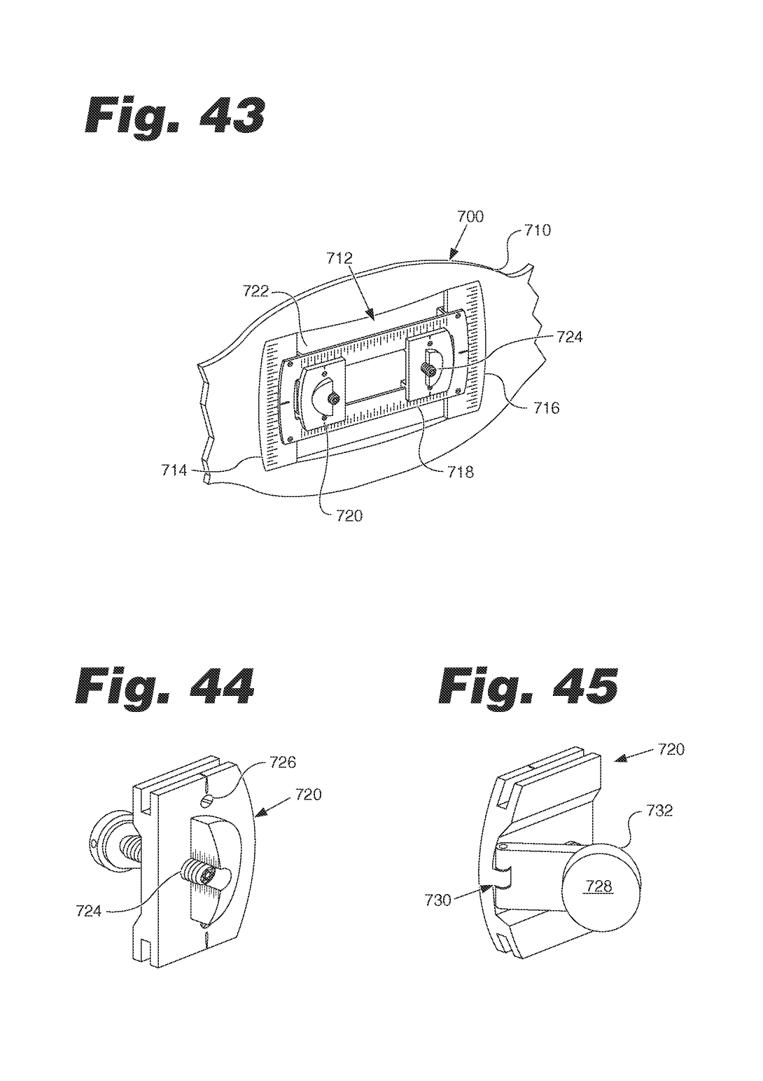

FIG. 43 is a back perspective view of a treatment head module housing in a nineteenth embodiment. The treatment head module housing 700 includes a frame 710, an opening 712, a first rail 714, a second rail 716, and a third rail 718. Transducer holders 720 are slidably attached to the third rail 718. In some embodiments, the rails 714, 716, 718 may include tick marks 722. The transducer holder 720 or the third rail 718 may include a locking mechanism 726. The third rail 718 may be adjusted in a proximal/distal direction and locked in place. Further, the transducer holders 720 may be adjusted in a medial/lateral direction.

FIGS. 44-45 illustrate a transducer holder in a ninth embodiment. The transducer holder 720 may include a shaft 724, and the shaft 724 may be threaded. In some embodiments, the transducer holder 720 includes a transducer mount 728. The transducer mount 728 may be pivotally attached through the use of a hinge 730. A transducer 732 may be connected to the transducer mount 728. Alternatively, the transducer 732 may be directly mounted on the transducer holder 720. In the embodiment depicted in FIG. 45, the shaft 724 is rotated to push against the transducer mount 728, the transducer mount 728 pivots to adjust the angle of the transducer 732.

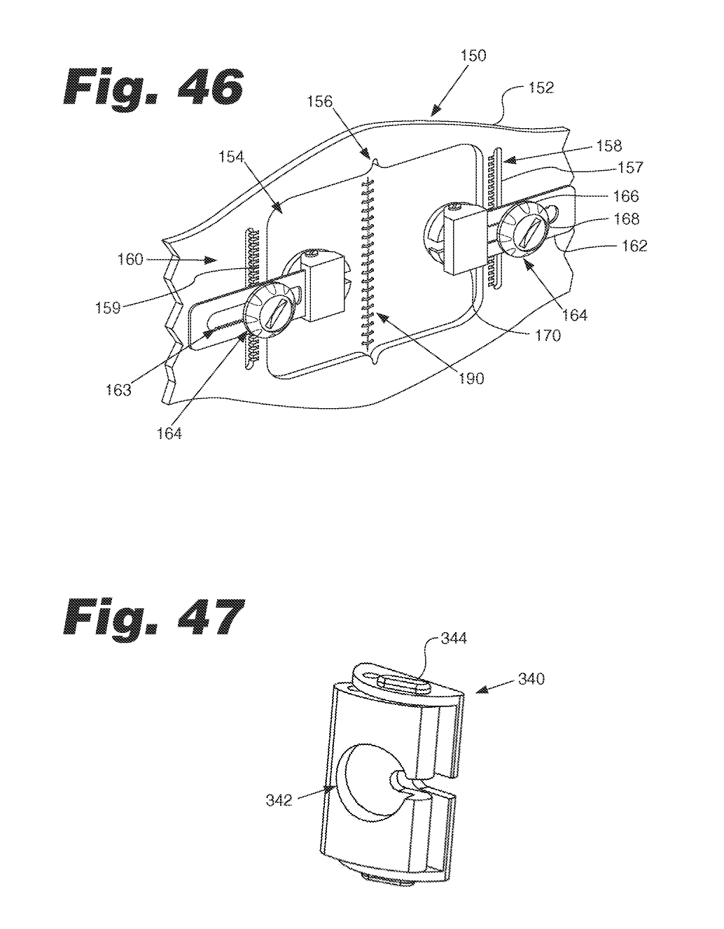

FIG. 46 is a back perspective view of a treatment head module housing in a twentieth embodiment. The treatment head module housing 150 includes a frame 152 and an opening 154. The frame 152 may include a first rail 158 and a second rail 160. The treatment head module housing 150 may include one or more yokes 162. Each yoke 162 may include gear teeth 163. The treatment head module housing 150 may include one or more alignment features 156. The alignment features 156 may be aligned with a patient's incision or scar 190. Each rail 158, 160 may include one or more slots 157. Each slot 157 may include one or more medial/lateral grooves, channels, or slots 159. A knob assembly 164 may connect each yoke 162 to each rail portion 158, 160. Each knob 164 may include an outer dial 166 and an inner dial 168. A swivable transducer holder 170 may be mounted to each yoke 162. The outer dial 166 includes teeth (not shown) to engage the teeth 163. The outer dial 166 may be rotated to move yokes 162 toward or away from the rails 158, 160. The inner dial 168 may include a fastener portion (not shown) adapted to engage a friction plate (not shown). The friction plate may engage the horizontal slots 159. The yoke 162 may be adjusted proximally or distally and held at the selected location via the inner dial 168 and the friction plate.

FIG. 47 is a detailed perspective view a transducer holder in a tenth embodiment. The transducer holder 342 is connected to a swivel yoke 340 through the use of a pin 344. The swivel yoke has multiple pin hole locations such that an angle of the transducer holder 342 may be selected and locked in place via the pin 344.

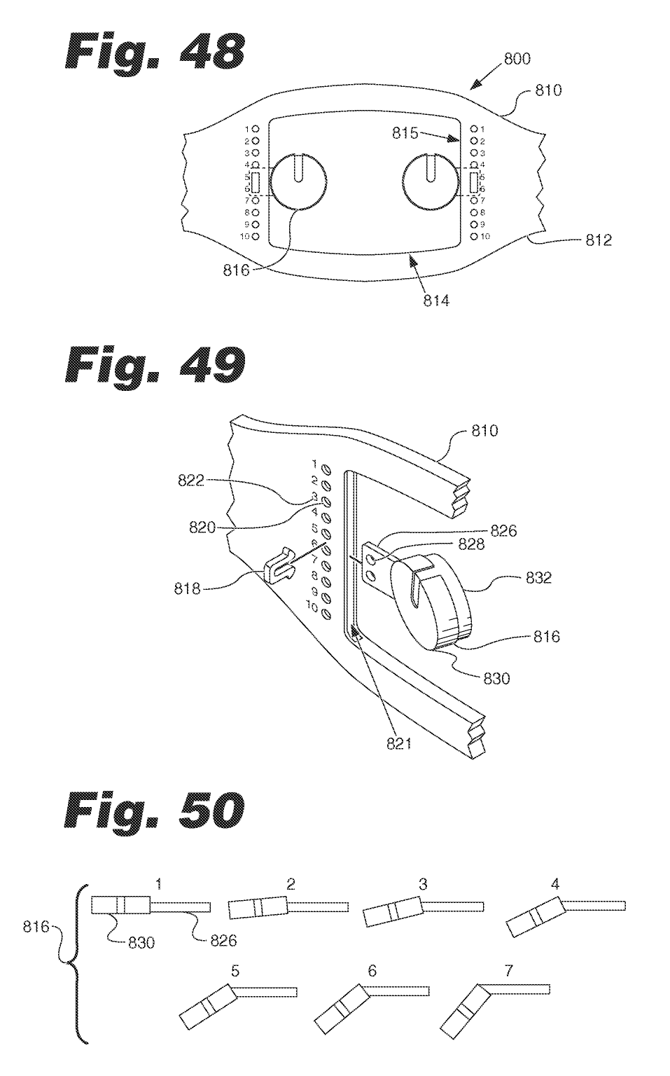

FIGS. 48-50 illustrate a treatment head module housing in a twenty-first embodiment. The treatment head module housing 800 includes a frame 810. A belt 812 may be attached to the frame 810. The frame 810 may include an opening 814. The frame 810 may include one or more rails 815. A transducer holder 816 may be connected to each rail 815. The transducer holder 816 may include a cup 830. The cup 830 is adapted to receive a transducer 832. As best seen in FIG. 49, a clip 818 may be used to connect the transducer holder 816 to the rail 815. The rail 815 may include one or more holes 820 and channel 821. The holes 820 may be marked with markings 822. The transducer holder 816 may include a tab 826 with holes 828. The tab 826 may be inserted into the channel 821 and the holes 820, 828 aligned such that the clip 818 may be inserted there through. As best seen in FIG. 50, the cup 830 may be angled relative to the tab 826. In this manner, a transducer holder 816 may be selected for the desired angle of the transducer 832 relative to the frame 810.

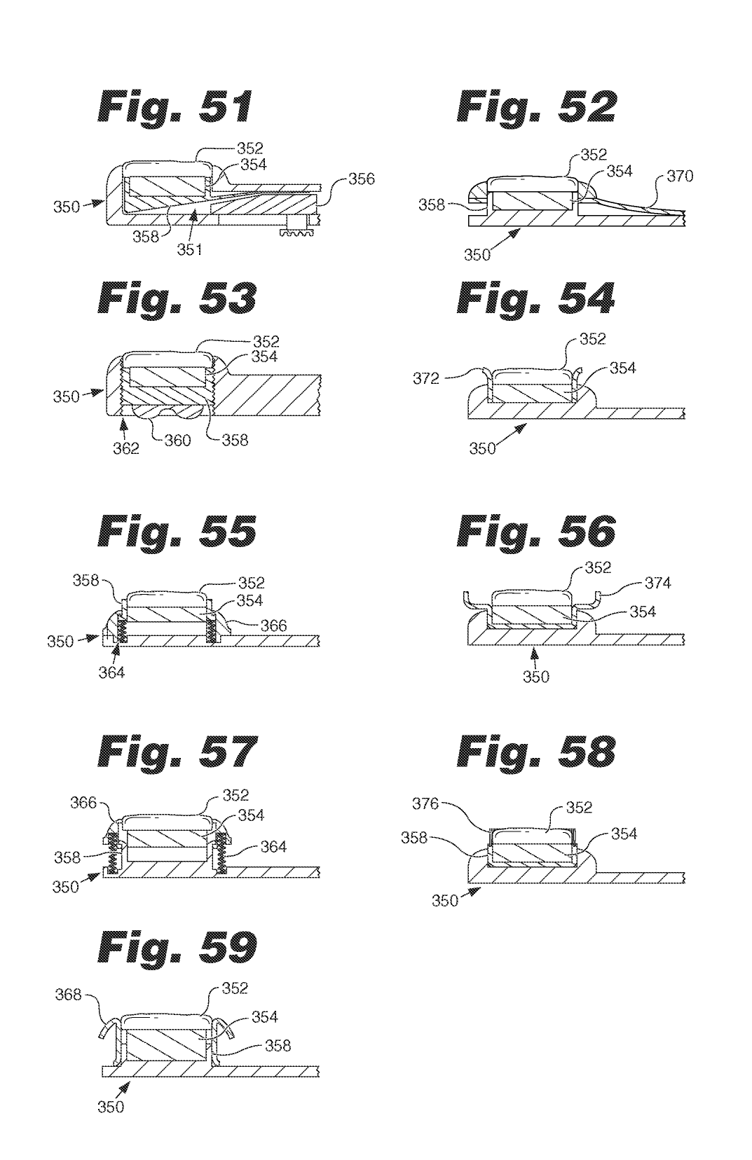

FIG. 51 is a sectional view of a yoke in a sixth embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 51, the cup 358 has a ramp portion 351. A sliding bar 356 engages the ramp portion 351 to affect the angle of the transducer 354. In other words, the sliding bar 356 may be moved in a medial/lateral direction to adjust the angle of the transducer 354.

FIG. 52 is a sectional view of a yoke in a seventh embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 52, the yoke 350 includes a hinged shroud 370.

FIG. 53 is a sectional view of a yoke in an eighth embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 53, the cup 358 forms a rotatable knob having a handle 360 and external threads. The yoke 350 includes threads 362, and the cup 358 is rotated in or out to vary the transducer 354 position.

FIG. 54 is a sectional view of a yoke in a ninth embodiment. The yoke 350 includes a cup 372. A transducer 354 and gel or a gel pack 352 are arranged in the cup 372. The cup 372 may be flexible and may be adapted to contain the gel 352.

FIG. 55 is a sectional view of a yoke in a tenth embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 55, the cup 358 is mounted on springs 364. A shroud 366 may used to contain the springs.

FIG. 56 is a sectional view of a yoke in an eleventh embodiment. The yoke 350 includes a stepped cup 374. A transducer 354 and gel or a gel pack 352 are arranged in the stepped cup 374. The stepped cup 372 may be flexible and may be adapted to contain the gel 352.

FIG. 57 is a sectional view of a yoke in a twelfth embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 57, a shroud 366 is mounted on springs 364.

FIG. 58 is a sectional view of a yoke in a thirteenth embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 58, flexible bristles 376 are attached to the cup 358.

FIG. 59 is a sectional view of a yoke in a fourteenth embodiment. The yoke 350 includes a cup 358. Gel or gel pack 352 and transducer 354 are connected to or rest upon the yoke 350. For example, the transducer 354 may sit within or be attached to the cup 358. In the embodiment depicted in FIG. 59, a shroud 368 surrounds the cup 358 and the gel 352.

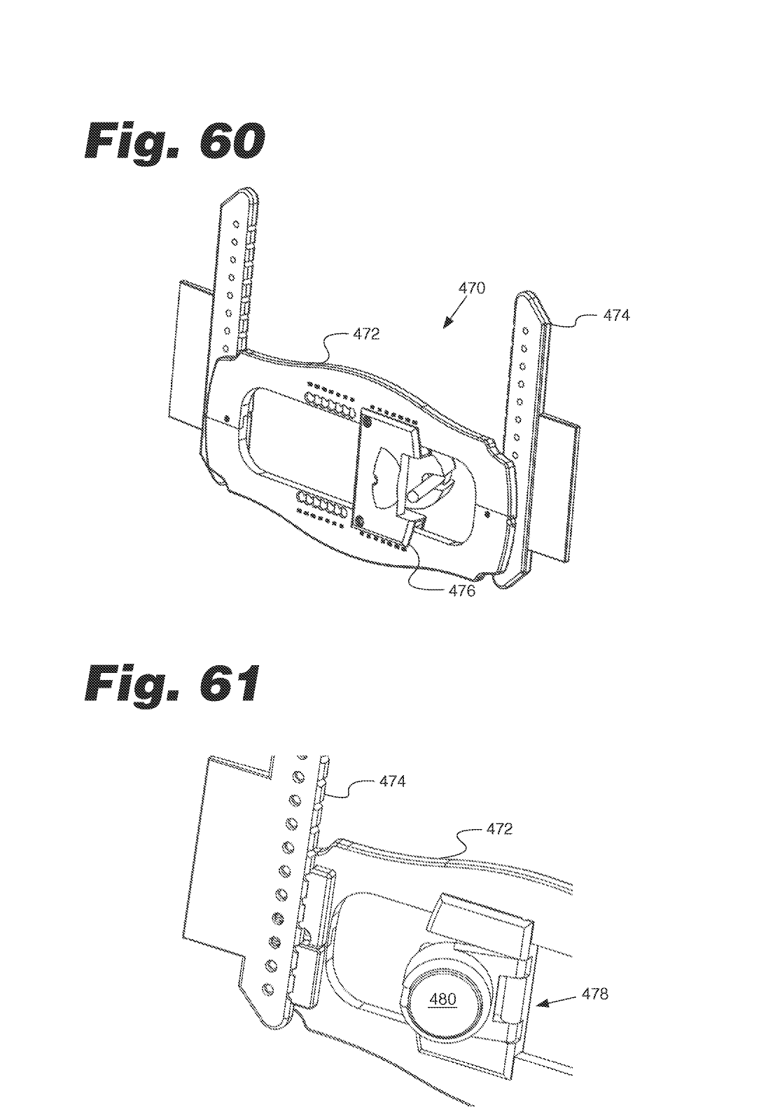

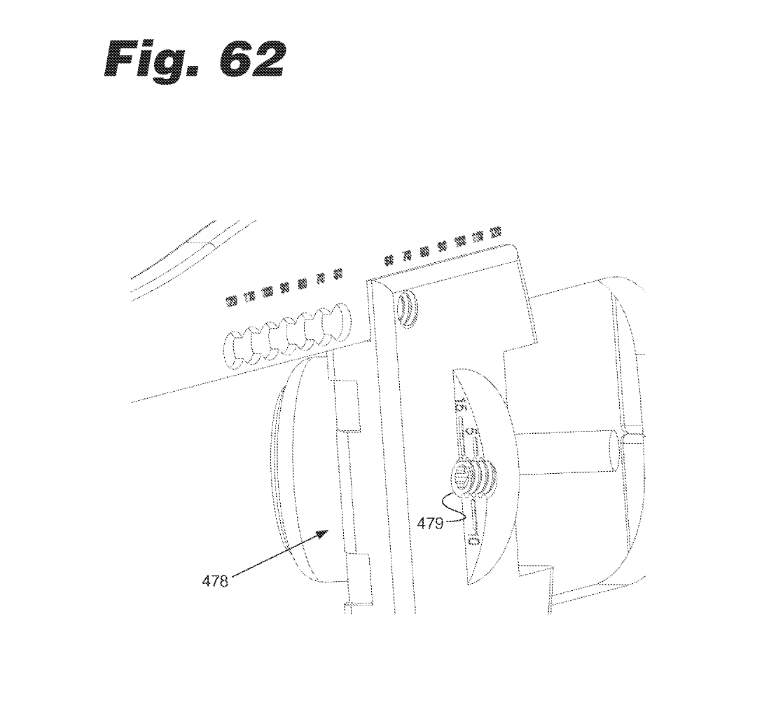

FIGS. 60-62 illustrate a treatment head module housing in a twenty-second embodiment. The treatment head module housing 470 includes a frame 472 and rails 474. A transducer holder 476 may be connected to the frame 472. In the depicted embodiment, the frame 472 is adapted to move in a proximal/distal direction and the transducer holder 476 is adapted to move in a medial/lateral direction. The treatment head module 470 may include one or more locking mechanism to hold the frame or transducer holder in place after adjustment. The transducer holder may include a hinge 478 to allow for angular adjustment of a transducer 480. The transducer holder 476 may include an adjustment screw 479 to adjust the angle of the transducer 480. The transducer holder 476 may include markings to indicate the angle of adjustment.

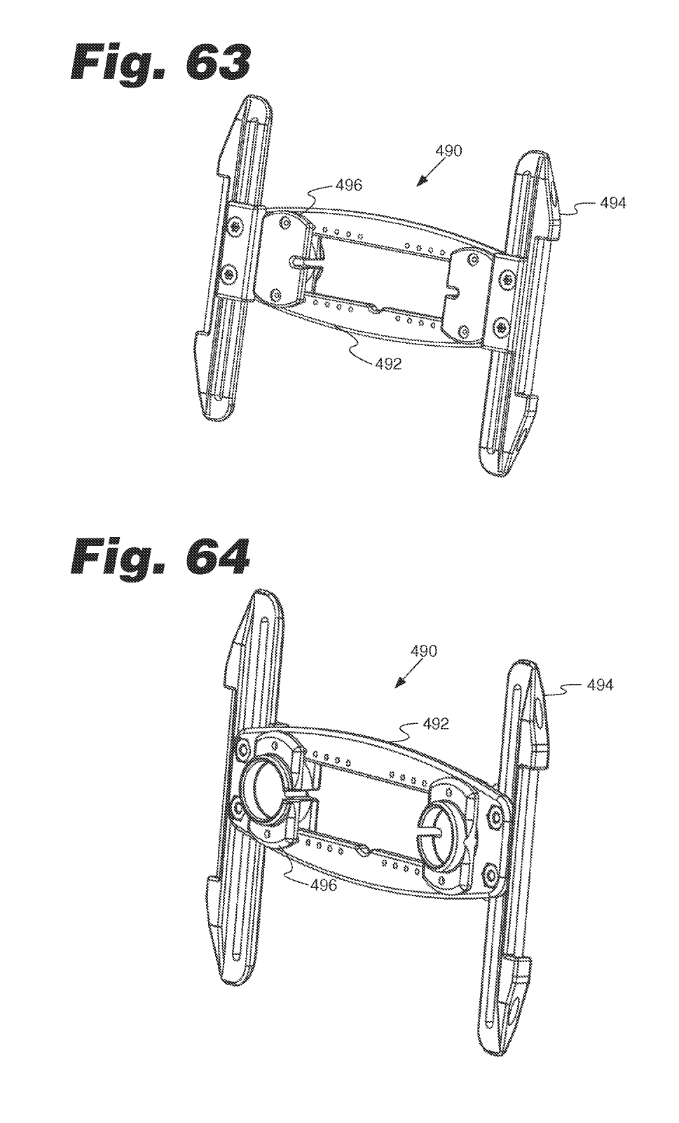

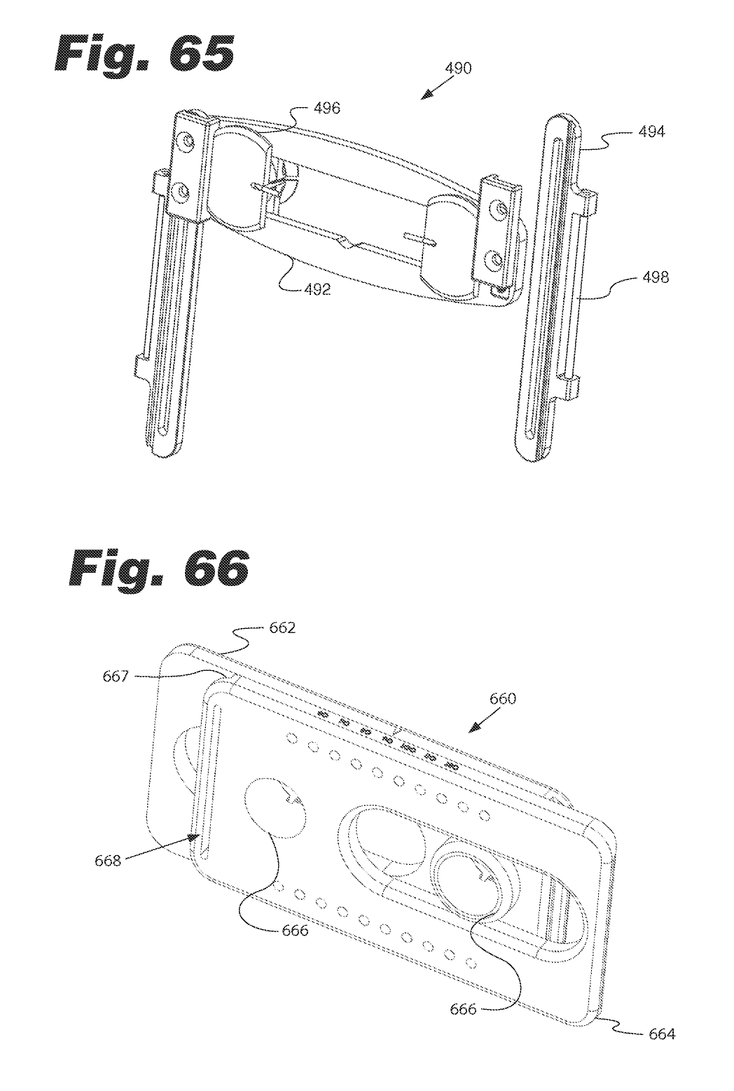

FIGS. 63-65 illustrate a treatment head module housing in a twenty-third embodiment. The treatment head module housing 490 includes a frame 492 and rails 494. A transducer holder 496 may be connected to the frame 492. In the depicted embodiment, the frame 492 is adapted to move in a proximal/distal direction and the transducer holder 496 is adapted to move in a medial/lateral direction. The treatment head module 490 may include one or more locking mechanism to hold the frame or transducer holder in place after adjustment. In some embodiments, a rod 498 may be attached to each rail 494 to provide structure for attaching a belt.

FIG. 66 is a detailed perspective view of a treatment head module housing in a twenty-fourth embodiment. The treatment head module housing 660 includes a first sliding member 662 and a second sliding member 664. Each sliding member 662, 664 includes a transducer holder 666 and a stepped edge 667. Each sliding member 662, 664 may include a belt attachment 668 for attaching a belt. The treatment head module housing 660 may be placed across a patient's back and adjusted in width by sliding the sliding members 662, 664 relative to one another. The treatment head module housing 660 may include markings to indicate a distance between transducer holders 666.

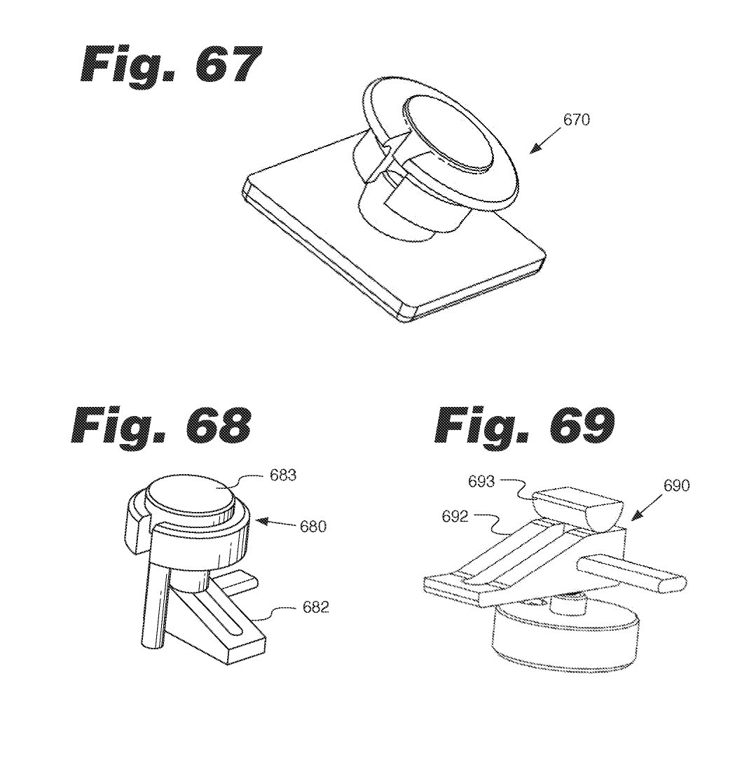

FIG. 67 is a detailed perspective view a transducer holder in an eleventh embodiment. The transducer holder 670 has an angular adjustment and a rotational adjustment. After adjustment, it can be locked in place. This is similar to the embodiment shown in FIG. 10.

FIGS. 68-69 illustrate a transducer holder in a twelfth embodiment. The transducer holder 680 includes a cam 682 and a slider 683. The slider 683 moves along the cam 682 to move the transducer in or out. In the embodiment depicted in FIG. 69, the transducer holder 690 includes a cam 692 and a slider 693. The slider 693 moves along the cam 692 to move the transducer in or out.

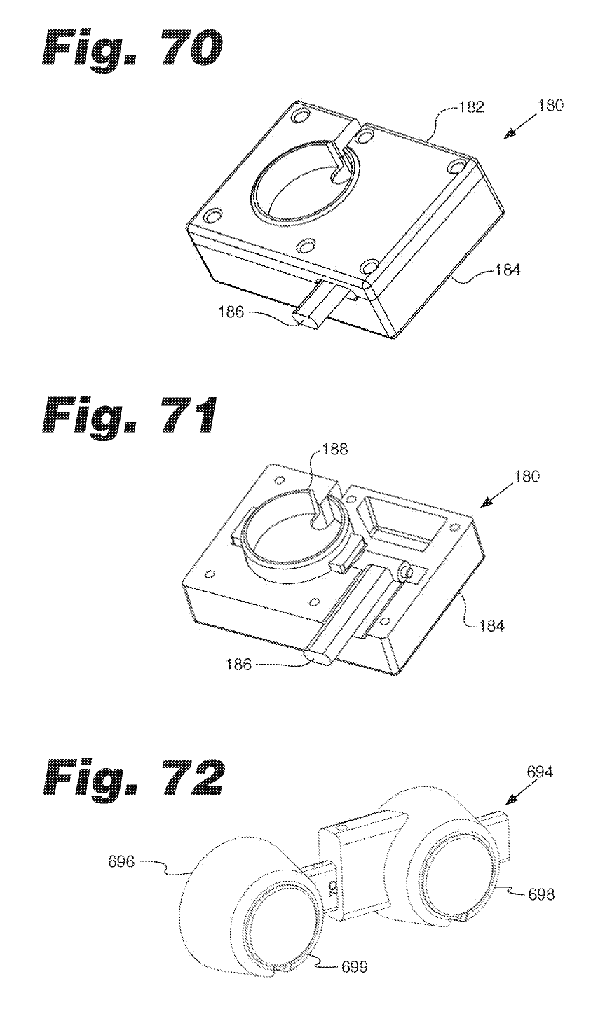

FIGS. 70-71 illustrate a transducer holder in a thirteenth embodiment. The transducer holder 180 includes a lid 182, a body 184, a handle 186, and a spring loaded cup 188. The cup 188 is biased against the spring and held in place through the use of the handle 186. The handle 186 may be moved to release the cup 188.

FIG. 72 is a detailed perspective view of a yoke in a sixteenth embodiment. The yoke 694 includes a first sliding member 696 and a second sliding member 698. Each sliding member 696, 698 includes a transducer holder 699. The yoke 694 may be placed across a patient's back and adjusted in width by sliding the sliding members 696, 698 relative to one another. The yoke 694 may include markings to indicate a distance between transducer holders 699.

FIG. 73 is a first belt configuration. In the depicted embodiment, there is an assembly 900 having a frame 910 and a belt 912, and the belt 912 is substantially centered with respect to the frame 910.

FIG. 74 is a second belt configuration. In the depicted embodiment, there is an assembly 920 having a frame 922 and a belt 924, and the belt 924 is attached to a lower portion of the frame 922 in proximal/distal direction but does not extend below the frame 922. In some embodiments, the frame 924 is extended proximally or distally to accommodate a wider range of patients. In the depicted embodiment, an upper portion of the frame 924 is extended in a proximal direction by about 63.5 millimeters.

FIG. 75 is third belt configuration. In the depicted embodiment, there is an assembly 930 having a frame 932 and a belt 934. The frame 932 has three or more slots 936 on each side. The belt 934 attaches to the upper two slots 936 for low fractures. Alternatively, the belt 934 attaches to the lower two slots 936 for high fractures, as is shown in FIG. 76. In the depicted embodiment, the belt 934 is asymmetric. In some embodiments, the belt 934 may be worn upside down or downside up to reach a lower part or an upper part of the lumbar spine.

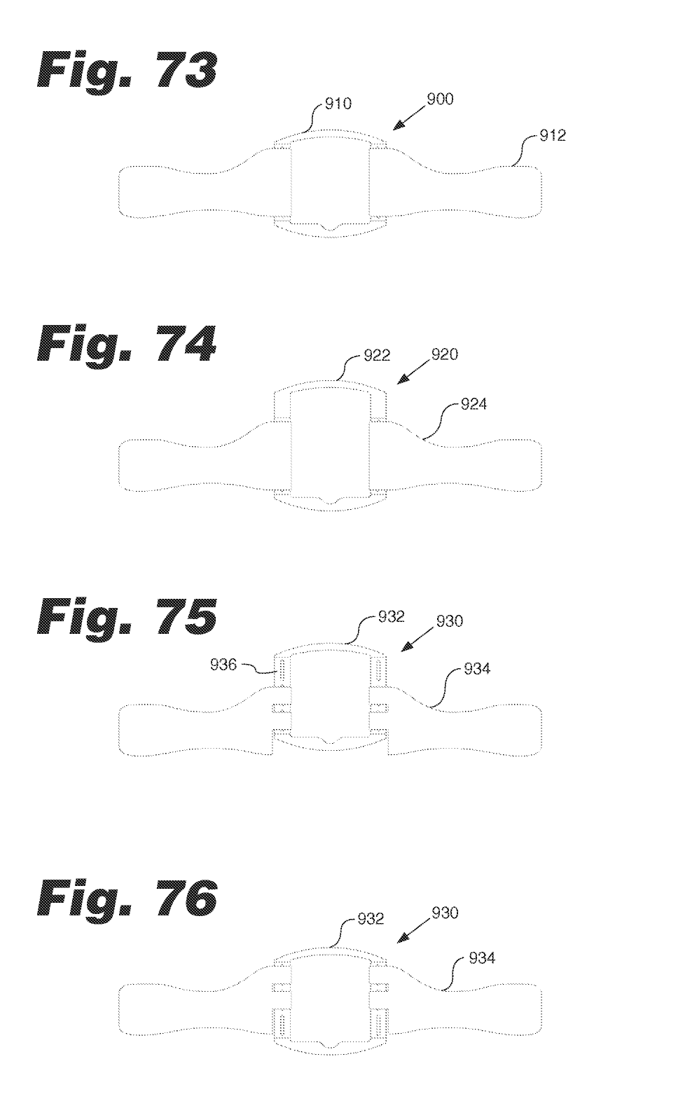

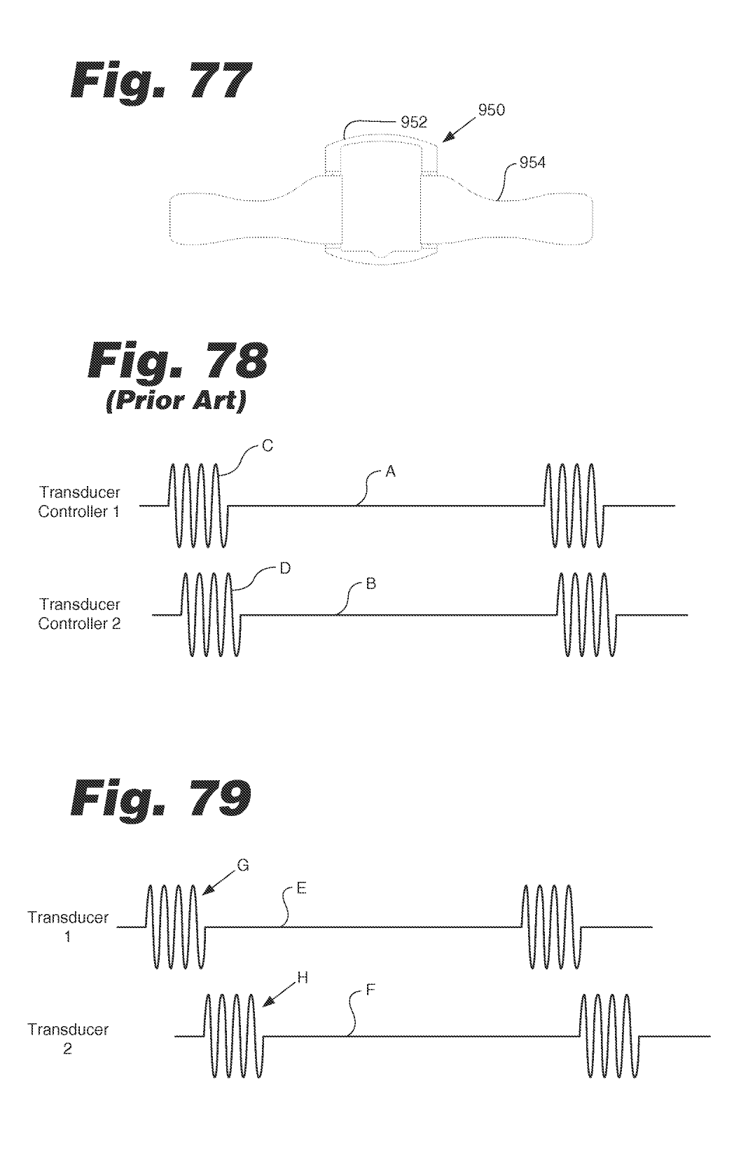

FIG. 77 is a fifth belt configuration. In the depicted embodiment, there is an assembly 950 having a frame 952 and a belt 954, and the belt 954 is attached to a lower portion of the frame 952 in proximal/distal direction but does not extend below the frame 952. The belt 954 may be worn right-side up for low fractures or upside-down for high fractures.

In some embodiments, suspenders (not shown) are attached to the belt 912, 924, 934, 954 to secure the frame 910, 922, 932, 952 at the appropriate height in relation to a fracture site. Further, in some embodiments, the frame 910, 922, 932, 952 is telescoping in a proximal/distal direction and may temporarily lock in place once the frame height is adjusted. Finally, the frame 910, 922, 932, 952 may be modular such that the height is adjusted during manufacturing based upon a selection of components.

FIG. 78 illustrates transducer signals of the prior art. In the depicted embodiment, an independent controller with two transducers results in one controller signal overlaying with the other controller signal. Referring to U.S. Pat. No. 5,762,616, which is incorporated by reference herein, the disclosed apparatus envisions a plurality of ultrasonic transducers that may all be activated at once for musculoskeletal injuries on the torso. In the prior art devices, there is a possibility that using independent controllers will cause the treatment signals to become asynchronous resulting in the possibility of treatment signal overlay, causing either destructive or constructive inference which has unknown effects.

FIG. 79 illustrates transducer signals of one particular embodiment. In the depicted embodiment, a single controller with two transducers synchronizes two or more signals without overlay.

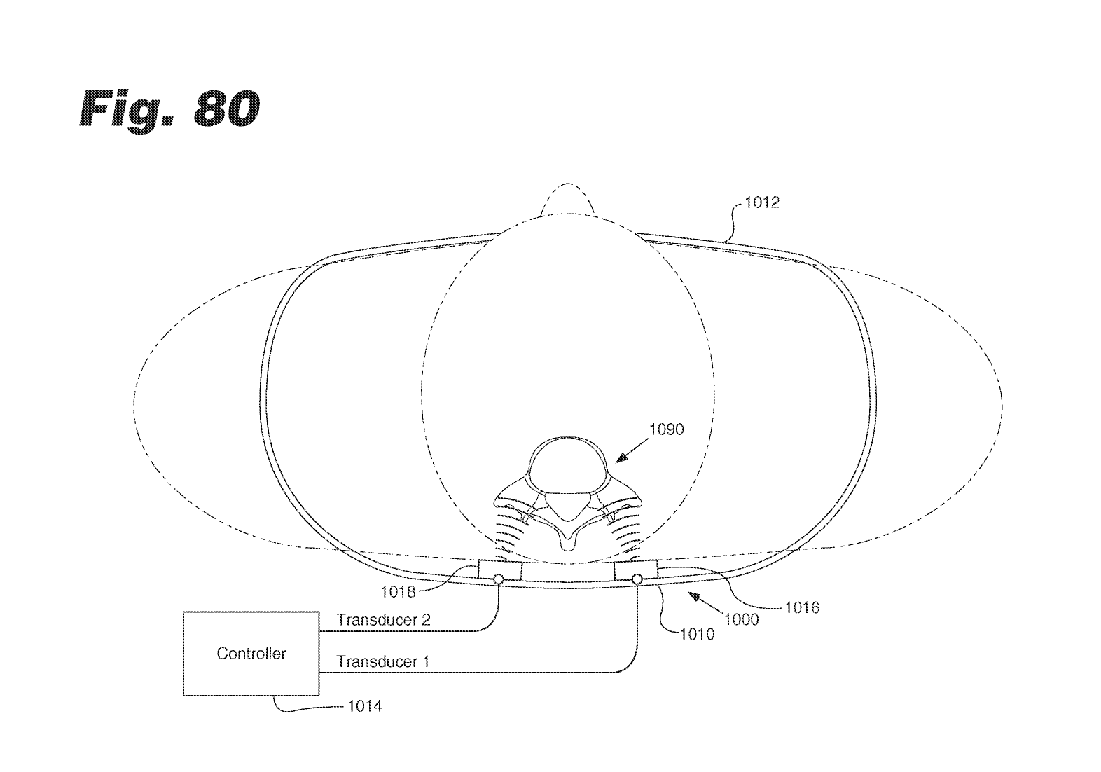

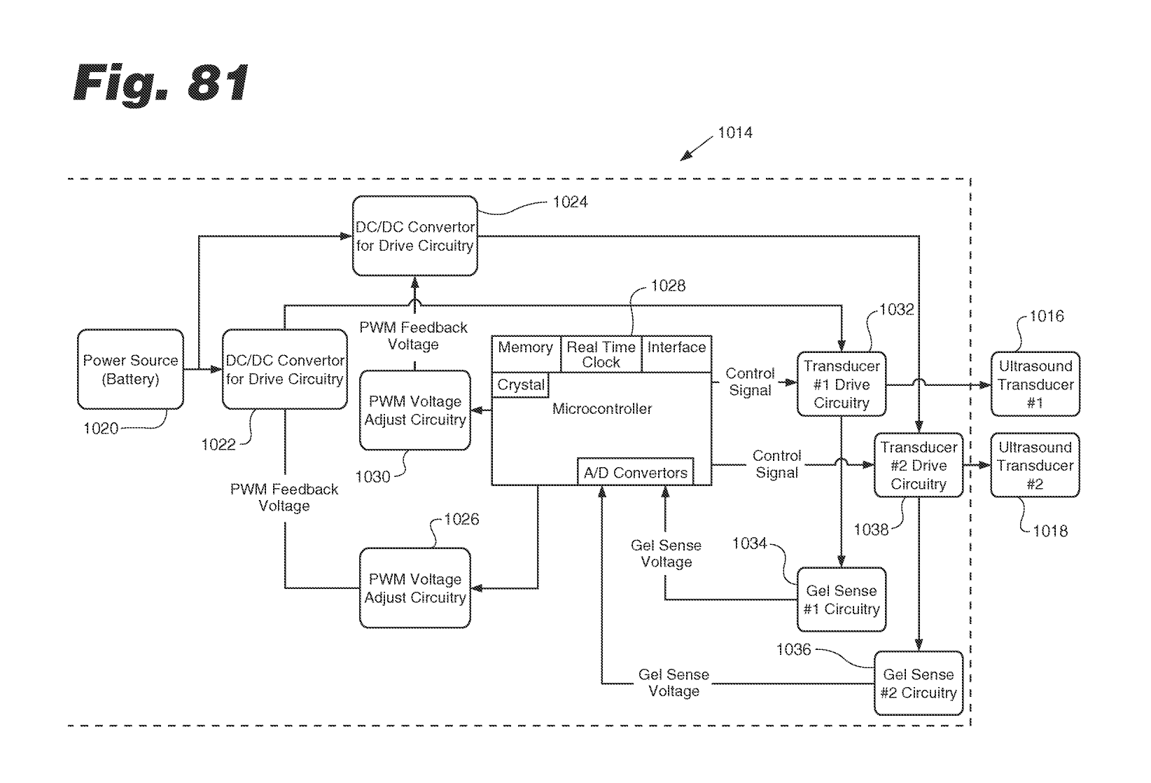

FIG. 80 illustrates a system for applying transducer signals to a spine 1090 without overlay. The system 1000 includes a frame 1010, a controller 1014, and at least two transducers 1016, 1018. The system 1000 may also include a belt 1012 to secure the frame 1010 to the patient. The use of a single controller 1014 ensures that the signals delivered stays synchronous. Further, a single controller 1014 with two or more transducers has a treatment time significantly less than a controller with one transducer.

FIG. 81 is a block diagram of the controller 1014. In the depicted embodiment, the controller 1014 includes a battery 1020, a first converter 1022, a second converter 1024, a first voltage adjust circuitry 1026, a microcontroller 1028, a second voltage adjust circuitry 1030, a first transducer drive circuitry 1032, a first gel sense circuit 1034, a second gel sense circuit 1036, and a second transducer drive circuitry 1038.

The components described herein may be made from a metal, polymer, or composite. The components may be made from a medical grade plastic. The components may be hypoallergenic. A cushioning material may be applied to some areas of the treatment head module housing to make it more comfortable for the user.

The apparatus described above may be used in conjunction with a method of treatment. The method of treatment may be used to significantly reduce the time to obtain partial or complete bone fusion. In the method, spinal fusion is performed on the patient by a health care provider. After a period of time, such as 1-4 weeks, the patient returns to the health care provider for fitting of a treatment head module housing to the patient. The health care provider may use x-ray images, palpitation, and/or measuring devices to locate the area of the spinal fusion and placement of the treatment head module housing. In some embodiments of the method, the treatment head module housing is placed with respect to the iliac crest such that it may be applied in a repeatable manner. After placement of the treatment head module housing, the position of the transducers may be adjusted relative to the housing and relative to the spine fusion area. In some embodiments, radiographic markers may be used to place the treatment head module housing and/or the transducers. Thereafter, the spine fusion area is treated with ultrasound on a regular basis for a period of time. For example, the spine fusion area may be treated daily with ultrasound for a period of 10-30 minutes. One particular example, the spine fusion area is treated for a period of 20 minutes per day.

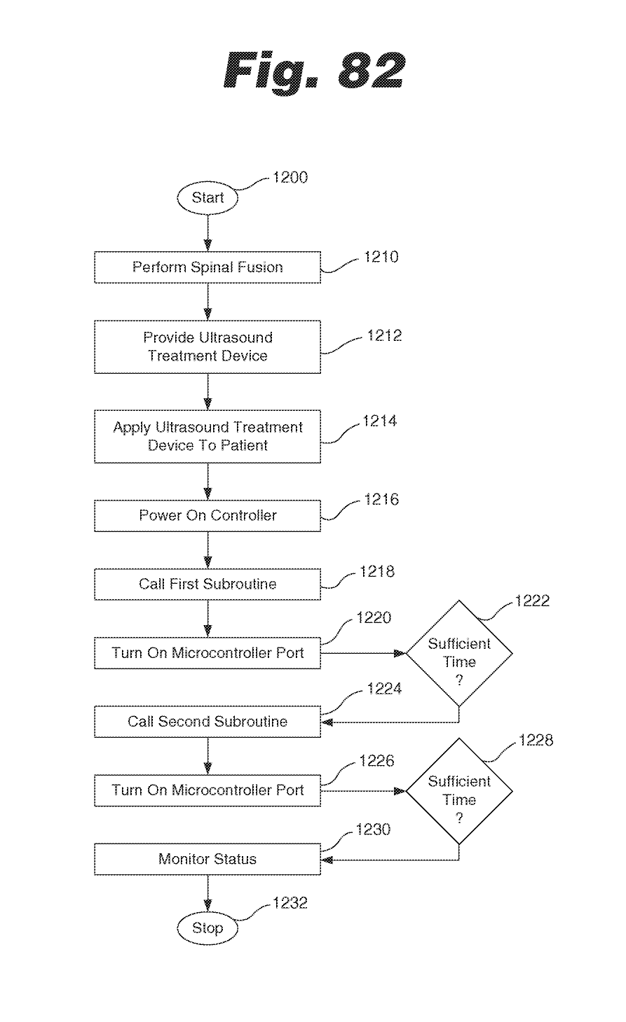

FIG. 82 illustrates a flowchart for a method of ultrasound application. The method starts in step 1200. In optional step 1210, spinal fusion is performed on a patient. In step 1212, there is provided an ultrasound treatment device, which may include the treatment head module housing. In step 1214, the ultrasound treatment device is applied to the patient. This step may include fitting the treatment head module housing to the patient and/or locating the treatment head module housing with respect to the iliac crest. The controller is engaged or powered up in step 1216. The controller may include a microprocessor. In step 1218, the microprocessor calls a first subroutine. In step 1220, the microprocessor turns on a first microprocessor port which engages a signal for a first transducer drive circuitry. In step 1222, there is a decision whether the microprocessor port has been engaged for a sufficient period of time. As an example, sufficient time may be as little as about 200 microseconds or about 300 cycles. If so, in step 1224 the microprocessor calls a second subroutine. Otherwise, the microprocessor continues to send a signal to the first transducer driver circuitry. In step 1226, the microprocessor turns on a second microprocessor port which engages a signal for a second transducer drive circuitry. In step 1228, there is a decision whether the microprocessor port has been engaged for a sufficient period of time. As an example, sufficient time may be as little as about 200 microseconds or about 300 cycles. If so, in step 1230 the microprocessor proceeds to monitor status of various functions, which may include such things as gel status, battery status, etc. Otherwise, the microprocessor continues to send a signal to the second transducer driver circuitry. In some embodiments, the process loops back to step 1218. Various steps in the method may be looped or repeated to obtain an acceptable period of application. The process ends in step 1232. As an example, the process may automatically end after a period of time, such as twenty minutes.

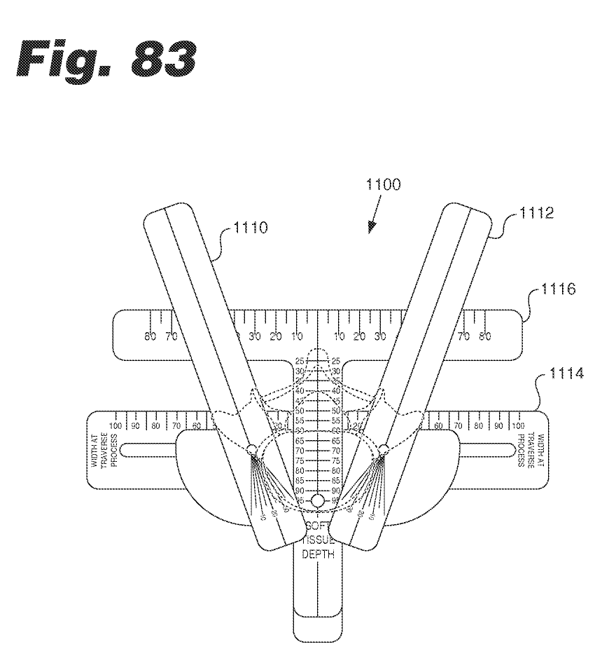

FIG. 83 is an instrument for selecting transducer placement. A spine goniometer 1100 may be used in conjunction with a CT-image or MRI scan to set an angle for each transducer. The spine goniometer 1100 has two pivotable arms 1110, 1112, a stationary arm 1114, and a slideable arm 1116. The spine goniometer 1100 is placed over the image and adjusted to match the bony anatomy and the fixation components. Thereafter, the angle is read and used to select the angle of the transducers.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Features of the various embodiments may be combined in any desirable manner. Thus, the breadth and scope of the present invention should not be limited by any of the above-described exemplary embodiments, but should be defined only in accordance with the following claims appended hereto and their equivalents.

1. An ultrasound delivery system for use in accelerating bone formation in a subject, the system comprising a subject attachment structure having a subject-facing surface which is configured to substantially contour to a region of the subject to which ultrasound is to be delivered, the subject attachment structure comprising;
a pair of ultrasound transducer holders located on the subject-facing surface, each holder capable of receiving an ultrasound transducer,
a belt attachment region.

2. An ultrasound delivery system according to claim 1, wherein the subject attachment structure is provided with a mechanism for altering at least one of the superior/inferior, the medial/lateral, or the anterior/posterior position of at least one transducer holder of the pair relative to the region of the subject to which the subject attachment structure is applied.

3. An ultrasound delivery system according to claim 2, wherein the transducer holders are removably locatable on the subject attachment structure.

4. An ultrasound delivery system according to claim 3, wherein the subject attachment structure is provided with an array of transducer holder positioning holes to which the transducer holders are securable.

5. An ultrasound delivery system according to claim 2, wherein at least one transducer holder of the pair is carried on a transducer holder carrying member and wherein the transducer holder carrying member is associated with and moveable along a track which extends at least partially across the subject-facing surface.

6. An ultrasound delivery system according to claim 5, wherein each of the transducer holders of the pair is carried on the transducer holder carrying member.

7. An ultrasound delivery system according to claim 6, wherein each transducer holder extends from the transducer holder carrying member in an opposing direction.

8. An ultrasound delivery system according to claim 5, wherein the transducer holder carrying member extends between two substantially parallel tracks.

9. An ultrasound delivery system according to claim 5, wherein each of the transducer holders of the pair is carried on a separate transducer holder carrying member, and wherein each transducer holder carrying member is associated with and moveable along a track.

10. An ultrasound delivery system according to claim 9, wherein the transducer holder carrying members are movable along the same track.

11. An ultrasound delivery system according to claim 10, wherein the transducer holder carrying members are independently moveable along the track.

12. An ultrasound delivery system according to claim 9, wherein each transducer holder carrying member is associated with a separate track.

13. An ultrasound delivery system according to claim 12, wherein the separate tracks are substantially parallel.

14. An ultrasound delivery system according to claim 12, wherein the separate tracks are substantially perpendicular.

15. An ultrasound delivery system according to claim 5, wherein the transducer holder carrying member is slidably attachable to the track.

16. An ultrasound delivery system according to claim 5, wherein the subject attachment structure is provided with a peripheral frame and wherein the track extends between opposing edges of the frame.

17. An ultrasound delivery system according to claim 16, wherein the track forms opposing edges of the frame.

18. An ultrasound delivery system according to claim 5, wherein the track is a rail which extends outwardly from the subject-facing surface.

19. An ultrasound delivery system according to claim 5, wherein the transducer holder or at least one transducer holder of a pair is moveable on the transducer holder carrying member.

20. An ultrasound delivery system according to claim 19, wherein the transducer holder is slidably moveable on the transducer holder carrying member.

21. An ultrasound delivery system according to claim 5, wherein the transducer holders are capable of being adjusted in an anterior/posterior direction relative to the subject when the subject attachment structure is in situ.

22. An ultrasound delivery system according to claim 5, wherein at least one of the transducer holder or the transducer holder carrying member is provided with a mechanism to enable at least of one of the angular or rotational adjustment of the transducer holder.

23. An ultrasound delivery system according to claim 22, wherein a locking mechanism is provided to lock the transducer holder in a specific orientation.

24. An ultrasound delivery system according to claim 22, wherein the angle of the transducer holder can be adjusted to be from about 1° to about 50° relative to the subject-facing surface.

25. An ultrasound delivery system according to claim 24, wherein the angle is from about 1° to about 35° relative to the subject-facing surface.

26. An ultrasound delivery system according to claim 1, wherein the transducer holder comprises a dish or cup portion adapted to receive a transducer.

27. An ultrasound delivery system according to claim 1, wherein a gel retaining cup is associated with the transducer holder.

28. An ultrasound delivery system according to claim 1, wherein the subject attachment structure comprises at least two pairs of transducer holders.

29. An ultrasound delivery system according to claim 1, wherein the subject attachment structure comprises an alignment feature for use in aligning the subject attachment structure with an anatomical structure and/or a marking on the subject.

30. An ultrasound delivery system according to claim 29, wherein the anatomical structure is a vertebrae.

31. An ultrasound delivery system according to claim 30, wherein the marking on the subject is an incision mark, a line of sutures or a scar.

32. An ultrasound delivery system according to claim 1, wherein the subject attachment structure is provided with at least one window.

33. An ultrasound delivery system according to claim 5, wherein measurement markings are provided adjacent to the track.

34. An ultrasound delivery system according to claim 1, wherein the subject attachment structure is configured to substantially contour to the torso.

35. An ultrasound delivery system according to claim 1, wherein at least an area of the subject attachment portion is made of a flexible material to enable the subject attachment structure to conform to the body.

36. An ultrasound delivery system according to claim 35, wherein the area of the subject attachment structure, which in use aligns with the vertebral column, is made of a substantially flexible material.

37. An ultrasound delivery system according to claim 36, wherein the area of the subject attachment structure which aligns with the vertebral column acts as a hinge.

38. An ultrasound delivery system according to claim 1, wherein a belt attachment region is provided on at least two opposing sides of the subject attachment structure.

39. An ultrasound delivery system according to claim 1, wherein the belt attachment region comprises a slot through which a portion of a belt is passable.

40. A method of accelerating bone formation in a subject, the method comprising the steps of;
providing an ultrasound delivery system comprising a subject attachment structure having a subject-facing surface which is configured to substantially contour to a region of the subject to which ultrasound is to be delivered, the subject attachment structure comprising;
a pair of ultrasound transducer holders located on the subject-facing surface, each holder capable of receiving an ultrasound transducer,
a belt attachment region;
(ii) introducing a ultrasound transducer into each transducer holder;
(ii) placing the subject-facing surface of the subject attachment structure against the treatment site;
(v) adjusting the position of at least one of the transducer holders in at least one of the medial/lateral, or the inferior/superior or the anterior/posterior position relative to the treatment site;
(vi) introducing a belt into the belt attachment region and securing the subject attachment structure at the treatment site.

41. A method according to claim 40, wherein the angular position of at least one transducer holder can be altered relative to the treatment site.

42. A method according to claim 40, wherein the method is used for accelerating bone formation in spinal indications.

43. A method according to claim 40, wherein the method is used to obtain partial or complete vertebral fusion.

44. A method according to claim 42, wherein the pair of transducer holders are positioned such that a transducer holder is located on either side of a vertebrae.

45. A method according to claim 40, wherein the subject attachment structure is placed at the iliac crest.

46. A method according to claim 40, wherein ultrasound is applied to the treatment site on a daily basis.

47. A method according to claim 46, wherein the ultrasound is applied for a period of from about 10 to about 30 minutes.

48. A method according to claim 40, wherein the subject is a mammal

49. A method according to claim 58, wherein the mammal is a human.

50. A method according to claim 48, wherein the subject is a non-human mammal

51. (canceled)
